(19) United States
(12) Patent Application Publication
(10) Pub. No.: US 2023/0242511 A1
(43) Pub. Date: Aug. 3, 2023

(54) FUSED PYRAZOLE AND IMIDAZOLE BASED COMPOUNDS AND USE THEREOF AS GLI1 INHIBITORS

(71) Applicant: GLIXOGEN THERAPEUTICS LTD., Ness Ziona (IL)

(72) Inventors: Yair FISHER, Avigdor (IL); William J. GREENLEE, Teaneck, NJ (US); Joel Moshe VAN GELDER, Jerusalem (IL)

(73) Assignee: GLIXOGEN THERAPEUTICS LTD., Ness Ziona (IL)

(21) Appl. No.: 17/925,003

(22) PCT Filed: May 14, 2021
(86) PCT No.: PCT/IL2021/050561

§ 371 Date: Nov. 13, 2022

Related U.S. Application Data
(60) Provisional application No. 63/024,767, filed on May 14, 2020.

(51) Int. Cl. C07D 403/04; C07D 471/04; C07D 519/00; C07D 487/04; A61P 25/00; A61P 29/00

(52) U.S. Cl. CPC C07D 403/04; C07D 471/04; C07D 519/00; C07D 487/04; A61P 25/00; A61P 29/00

(57) ABSTRACT

The present invention is directed to a composition and a method for use thereof, such as for the treatment and prevention of a neurological disorder or cancer in a subject.

### CROSS REFERENCE TO RELATED APPLICATIONS

This application claims the benefit of priority of U.S. Provisional Patent Application No. 63/024,767 filed May 14, 2020, entitled “FUSED PYRAZOLE AND IMIDAZOLE BASED COMPOUNDS AND USE THEREOF AS GiI1 INHIBITORS”, the contents of which are incorporated herein by reference in their entirety.

### FIELD OF THE INVENTION

The invention relates generally to the field of compositions comprising one or more fused pyrazole or fused imidazole derivatives and is directed to methods of using the same such as for treating a disease related to glioma-associated oncogene (Gli) expression.

### BACKGROUND

Hedgehog (Hh) signaling pathway plays a critical role in the initiation, proliferation, invasion, and metastasis of a wide variety of cancers. The Hh pathway is also implicated in the regulation and maintenance of cancer stem cells (CSCs), providing a link between the Hh signaling in the regulation of normal stem cells and its role in CSCs maintenance.

More recently, the involvement of Smo and Gli was also addressed in the context of myelin regeneration. Disorders of myelination can produce significant impairment in sensory, motor and other types of functioning when nerve signals reach their targets slowly, asynchronously, intermittently, or not at all. Disorders of myelination (e.g. demyelinating and/or dysmyelinating disorders) may affect both central nervous system (CNS) and peripheral nervous system (PNS) myelin, and are also associated with progressive loss of the axons which further contributes to neurological impairment. Disorders of myelination include inter alia multiple sclerosis (MS), leukodystrophies, the Guillain Barre Syndrome, and the Charcot Marie Tooth inherited peripheral neuropathies.

Hh signaling involves inter alia activation of the Gli family of proteins (also known as zinc finger transcription factors).

Vertebrates have at least three distinct Gli proteins, Gli1, Gli2, and Gli3 (glioma-associated oncogene 1, 2, and 3). Gli proteins participate in the final step of the Hh/Gli signaling pathway, and they regulate several genes, including those that are related to cell cycle control and Hh/Gli signaling. Gli1 acts as a transcriptional activator, whereas Gli2 and Gli3 act as both activators and repressors. Of the three Gli proteins, Gli1 expression is considered a sensitive readout for, and an indicator of the highest levels of Shh signaling.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

In one aspect of the invention, there is a compound or a salt thereof, wherein the compound is represented by or comprises Formula VIIIa:

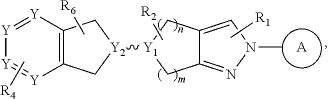

wherein: A comprises hydrogen, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof; each R₁, R₂ and R₆ represents a substituent independently comprising halogen, —NO₂, —CN, optionally substituted C₁-C₆ alkyl, C₁-C₆ haloalkyl, —NH₂, —NH(C₁-C₆ alkyl), —N(C₁-C₆ alkyl)₂, carbonyl, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONN—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, and optionally substituted C₃-C₈ cycloalkyl or a combination thereof, or each R₁, and R₂ independently represents hydrogen; R₄ represents a substituent comprising hydrogen, halogen, —NO₂, —CN, C₁-C₆ alkyl optionally substituted with one or more R₅, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, optionally substituted C₃-C₈ cycloalkyl, optionally substituted heterocyclyl, optionally substituted aryloxy, optionally substituted heteroaryloxy, optionally substituted cycloalkyloxy, 2-hydroxy-3-methoxypropoxy, (2-methoxyethoxy)methyl, and 2-(3-(but-3-yn-1-yl)-3H-diazirin-3-yl)ethoxy or a combination thereof; each R independently comprises hydrogen, C₁-C₆ alkyl, tolyl, optionally substituted phenyl, optionally substituted benzyl or a combination thereof; Y, Y₁ and Y₂ independently comprises CH, C, N, or NH; and each m and n is independently 1 or 2.

In some embodiments, the compound is represented by or comprises Formula VIIIa:

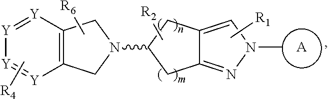

or formula VIIIb:

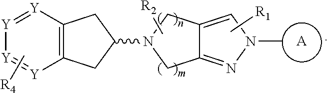

In some embodiments, A comprises C₅-C₆ aryl optionally substituted with one or more R₃, C₅-C₆ heteroaryl optionally substituted with one or more R₃, or C₄-C₈ cycloalkyl optionally substituted with one or more R₃ or a combination thereof; wherein R₃ is selected from the group comprising hydrogen, halogen, —NO₂, —CN, optionally substituted C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, and optionally substituted C₃-C₈ cycloalkyl or a combination thereof.

In some embodiments, A comprises any of:

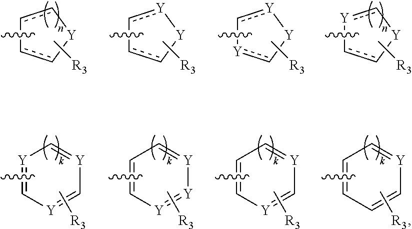

wherein k is 0 or 1 and n is 1 or 2. In some embodiments, compound is stable in an aqueous solution for at least 1 hour.

In another aspect of the invention, there is a pharmaceutical composition, comprising the compound of the invention and a pharmaceutically acceptable carrier.

In some embodiments, the pharmaceutical composition is for use in the inhibition of glioma-associated oncogene (Gli) cell activity. In some embodiments, the pharmaceutical composition is for use in the inhibition of mitosis in a cell expressing a glioma-associated oncogene (Gli). In some embodiments, the cell is a cancerous cell or a nerve cell. In some embodiments, Gli is Gli 1.

In another aspect of the invention, there is a method for preventing or treating a disease or a disorder associated with an abnormal expression of Gli in a subject, comprising administering to the subject the pharmaceutical composition of the invention, thereby preventing or treating a disease or a disorder associated with an abnormal expression of Gli in a subject.

In another aspect of the invention, there is a method for preventing or treating cancer in a subject, comprising administering to the subject the pharmaceutical composition of the present invention, wherein the cancer is characterized by comprising a cancerous cell expressing Gli or over-expressing Gli, thereby preventing or treating cancer in a subject.

In some embodiments, the method comprises the step of selecting the subject as: (a) afflicted with a disease or a disorder comprising an abnormal expression of Gli; or (b) afflicted the cancerous cell expressing Gli or abnormal expressing of Gli.

In some embodiments, the disease or the disorder comprises a neurological disorder or cancer.

In some embodiments, the cancer is selected from the group comprising: breast cancer, pancreatic cancer, colon cancer, lung cancer, rhabdomyosarcoma, basal-cell carcinoma, glioblastoma, medulloblastoma, leukemia, prostate cancer, skin cancer, lymphoma, esophageal cancer, ovarian cancer, thyroid cancer, osteosarcoma, liver cancer, multiple endocrine neoplasia, gastrointestinal cancer, or mesothelioma.

In some embodiments, the neurological disorder is selected from the group comprising: multiple sclerosis, central pontine myelinolysis, acute disseminated encephalomyelitis, progressive multifocal leukoencephalopathy, subacute sclerosing panencephalitis, post-infectious encephalomyelitis, chronic inflammatory demyelinating polyneuropathy, Devic's disease, Balo's concentric sclerosis, the leukodystrophies, optic neuritis, transverse myelitis, cerebral palsy, spinal cord injury, age-associated myelin deficiency, Alzheimer's Disease, and acquired and inherited neuropathy in the peripheral nervous system.

In some embodiments, the preventing or the treating comprises inhibiting Gli function in the cell. In some embodiments, the Gli is Gli1.

In some embodiments, the administering is by an oral administration, a topical administration, a systemic administration or any combination thereof.

In addition to the exemplary aspects and embodiments described above, further aspects and embodiments will become apparent by reference to the figures and by study of the following detailed description.

### BRIEF DESCRIPTION OF THE DRAWINGS

FIGS. 1A-B are graphs showing improvements in clinical outcome of mice treated with Gli1 inhibitor in the EAE mouse model. Bars represent Mean±SEM, Statistical analysis was performed using Dunnett's multiple comparisons test, n=15 for compound 6-treated groups and n=20 for vehicle-treated group (FIG. 1A) and bars represent Mean±SEM, Statistical analysis was done using 2-way ANOVA with Tukey's multiple comparison test. * shows difference from vehicle-treated animals. #shows difference between Glx-077 and Glx-088P3-treated animals. N=12 for all groups except for dexamethasone which had n=5 (FIG. 1B).

### DETAILED DESCRIPTION

In one aspect of the invention disclosed herein, there is a compound of Formula

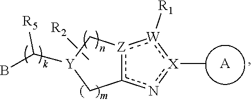

wherein each B and A independently comprises hydrogen, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof; wherein each R₁, and R₂ represents a substituent independently comprising halogen, —NO₂, —CN, carbonyl, optionally substituted C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, and optionally substituted C₃-C₈ cycloalkyl or a combination thereof, or each R₁, and R₂ independently represents hydrogen; wherein each R₅ independently comprises hydrogen, deuterium, C₁-C₆ alkyl, C₁-C₆ haloalkyl, optionally substituted C₃-C₈ cycloalkyl, —CO₂R, —CN, —CONRR, or a combination thereof; wherein each R independently comprises hydrogen, C₁-C₆ alkyl, tolyl, optionally substituted phenyl, optionally substituted benzyl or a combination thereof; wherein each X, Z and Y independently comprises CH, C, N, or NH; W comprises CH, C, [symbol] N, NH, S or O wherein represents a single or a double bond; wherein each m and n is independently 1 or 2; wherein k is between 0 and 5; and wherein if and if Y and W are N, and X and Z are C, then k is 1 and R₁ represents the substituent.

In one embodiment, provided herein is a composition comprising a compound of Formula I and a carrier. In one embodiment, provided herein a pharmaceutically acceptable salt of a compound of the invention such as but not limited to the compound of any of the Formulae disclosed herein.

In some embodiments, the compound of the invention is a pharmaceutical grade compound. In some embodiments, the pharmaceutical composition of the invention comprises one or more of the compounds disclosed herein, wherein the compounds are pharmaceutical grade compounds. In some embodiments, the compound of the invention refers to or further comprises a pharmaceutically acceptable salt thereof. In some embodiments, the composition of the invention comprises the compound of the invention, a pharmaceutically acceptable salt thereof or both. In some embodiments, any compound of the invention such as the compound of any of the Formulae disclosed herein, a pharmaceutically acceptable salt thereof or both is stable in an aqueous solution for at least one hour. In some embodiments, any compound of the invention such as but not limited to the compound of Formula I, a pharmaceutically acceptable salt thereof or both is stable in an aqueous solution for at least 2 hours, at least 5 hours, at least 10 hours, at least 24 hours, at least 36 hours including nay range or value therebetween.

In one aspect of the invention disclosed herein, there is a compound of Formula VIIIa:

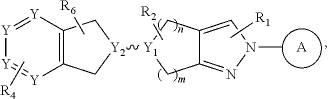

wherein A comprises hydrogen, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof; each R₁, R₂ and R₆ represents a substituent independently comprising halogen, —NO₂, —CN, optionally substituted C₁-C₆ alkyl, C₁-C₆ haloalkyl, —NH₂, —NH(C₁-C₆ alkyl), —N(C₁-C₆ alkyl)₂, carbonyl, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, and optionally substituted C₃-C₈ cycloalkyl or a combination thereof, or each R₁, and R₂ independently represents hydrogen; R₄ represents a substituent comprising hydrogen, halogen, —NO₂, —CN, C₁-C₆ alkyl optionally substituted with one or more R₅, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, optionally substituted C₃-C₈ cycloalkyl, optionally substituted heterocyclyl, optionally substituted aryloxy, optionally substituted heteroaryloxy, optionally substituted cycloalkyloxy, 2-hydroxy-3-methoxypropoxy, (2-methoxyethoxy)methyl, and 2-(3-(but-3-yn-1-yl)-3H-diazirin-3-yl)ethoxy or a combination thereof; each R independently comprises hydrogen, C₁-C₆ alkyl, tolyl, optionally substituted phenyl, optionally substituted benzyl or a combination thereof; Y, Y₁ and Y₂ independently comprises CH, C, N, or NH; and each m and n is independently 1 or 2.

In one embodiment, provided herein is a composition comprising a compound of Formula VIIIa and a carrier. In one embodiment, provided herein a pharmaceutically acceptable salt of a compound of the invention such as but not limited to the compound of Formula VIIIa.

In some embodiments, the composition comprises the compound of the invention, a pharmaceutically acceptable salt thereof or both. In some embodiments, any compound of the invention such as but not limited to the compound of Formula VIIIa, a pharmaceutically acceptable salt thereof or both is stable in an aqueous solution for at least one hour. In some embodiments, any compound of the invention such as but not limited to the compound of Formula VIIIa, a pharmaceutically acceptable salt thereof or both is stable in an aqueous solution for at least 2 hours. In some embodiments, any compound of the invention such as but not limited to the compound of Formula VIIIa, a pharmaceutically acceptable salt thereof or both is stable in an aqueous solution for at least 5 hours. In some embodiments, any compound of the invention such as but not limited to the compound of Formula VIIIa, a pharmaceutically acceptable salt thereof or both is stable in an aqueous solution for at least 10 hours. In some embodiments, any compound of the invention such as but not limited to the compound of Formula VIIIa, a pharmaceutically acceptable salt thereof or both is stable in an aqueous solution for at least 24 hours. In some embodiments, any compound of the invention such as but not limited to the compound of Formula VIIIa, a pharmaceutically acceptable salt thereof or both is stable in an aqueous solution for at least 36 hours.

In another aspect, the present invention provides a compound represented by Formula VIII: Formula VIIIa:

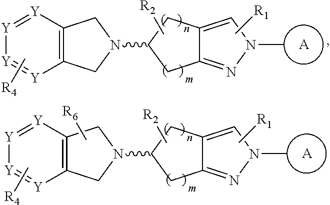

### or by Formula VIIIb:

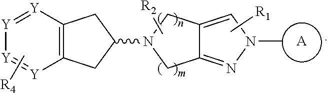

wherein R₁, R₂, R₄, A, m, and n are as described herein, and a wavy bond represent an R or an S enantiomer. In some embodiments, Y is nitrogen and each m and n is 1 or 2.

In some embodiments, A comprises C₅-C₆ aryl optionally substituted with one or more R₃, C₅-C₆ heteroaryl optionally substituted with one or more R₃, or C₄-C₈ cycloalkyl optionally substituted with one or more R₃ or a combination thereof wherein R₃ is selected from the group comprising hydrogen, halogen, —NO₂, —CN, optionally substituted C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, and optionally substituted C₃-C₈ cycloalkyl or a combination thereof.

In some embodiments, A comprises any of:

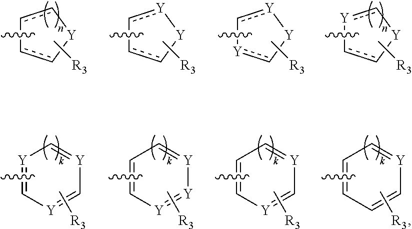

wherein k is 0 or 1 and n is 1 or 2.

In some embodiments, A comprises C₅-6 aryl optionally substituted with one or more R₃ or C₅-6 heteroaryl optionally substituted with one or more R₃.

In some embodiments, the R₂ represents a substituent selected from the group comprising hydrogen, halogen, —NO₂, —CN, optionally substituted C₁-C₆ alkyl, C₁-C₆ haloalkyl, —NH₂, —NH(C₁-C₆ alkyl), —N(C₁-C₆ alkyl)₂, carbonyl, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONN—OH, —CO₂H, —CO₂(C₁-C₆ alkyl), —SO₂R, —SO₂OR, —SO₂N(R)₂.

In some embodiments, R₁ represents a substituent independently comprising hydrogen, optionally substituted C₁-C₄ alkyl.

In another aspect, the present invention provides a compound represented by any of:

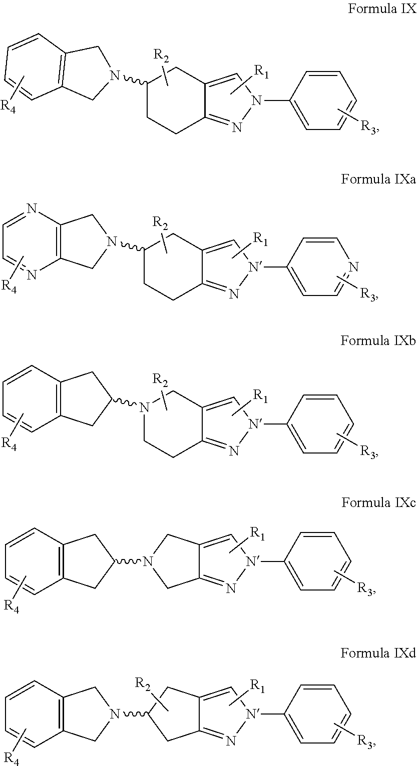

wherein R₁, R₃ and R₄ are as described hereinabove.

In another aspect, the present invention provides a compound represented by any of:

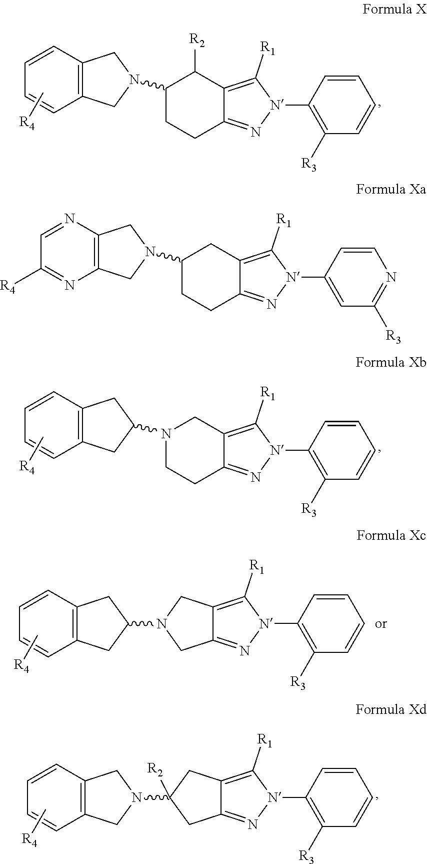

wherein R₁, R₃ and R₄ are as described hereinabove. In some embodiments, R₁ is methyl. In some embodiments, R₁ is —NH₂. In some embodiments, R₁ is —CN.

In some embodiments, each R₂, R₃ and R₄ independently represents a substituent selected from the group comprising halogen, carbonyl, —CN, —OMe, —OH, any combination thereof, or is absent.

In another aspect, the present invention provides a compound represented by Formula XI: or by Formula XIa:

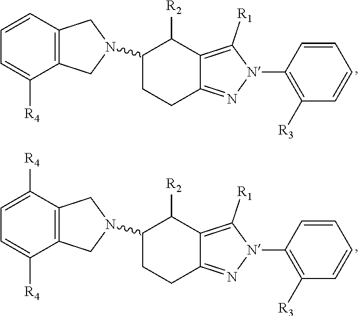

wherein R₁, R₃ and R₄ are as described hereinabove. In some embodiments, R₂ represents a substituent selected from the group comprising methyl, carbonyl, —OH, or is absent.

In some embodiments, each R₄ is independently selected from the group comprising hydrogen, halogen, —NO₂, —CN, C₁-C₆ alkyl optionally substituted with one or more R₅, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, optionally substituted C₃-C₈ cycloalkyl, optionally substituted heterocyclyl, optionally substituted aryloxy, optionally substituted heteroaryloxy, optionally substituted cycloalkyloxy, 2-hydroxy-3-methoxypropoxy, (2-methoxyethoxy)methyl, and 2-(3-(but-3-yn-1-yl)-3H-diazirin-3-yl)ethoxy or a combination thereof.

In some embodiments, R₄ is attached to a carbon atom or to a heteroatom. In some embodiments, R₄ attached to a carbon atom comprises hydrogen, halogen, —NO₂, —CN, C₁-C₆ alkyl optionally substituted with one or more R₅, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, optionally substituted C₃-C₈ cycloalkyl, optionally substituted heterocyclyl, optionally substituted aryloxy, optionally substituted heteroaryloxy, optionally substituted cycloalkyloxy, 2-hydroxy-3-methoxypropoxy, (2-methoxyethoxy)methyl, and 2-(3-(but-3-yn-1-yl)-3H-diazirin-3-yl)ethoxy or a combination thereof.

In some embodiments, R₄ attached to a carbon atom comprises hydrogen, halogen (e.g. Cl—, Br—, F—), —NO₂, —CN, C₁-C₆ alkyl optionally substituted with one or more R₅, —NH₂, —NH(C₁-C₆ alkyl), —N(C₁-C₆ alkyl)₂, —OH, or a combination thereof.

In some embodiments, each R₃ is independently selected from the group comprising hydrogen, halogen, —NO₂, —CN, optionally substituted C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, and optionally substituted C₃-C₈ cycloalkyl or a combination thereof.

In some embodiments, each R is independently selected from the group comprising a (C1-C8) alkyl, a (C3-C8) cycloalkyl, a (C1-C8) substituted alkyl, and hydrogen or is absent. In some embodiments, each R is independently selected from the group comprising a (C1-C5) alkyl, a (C1-C8) substituted alkyl, hydrogen or is absent.

In some embodiments, R comprises a (C1-C8) alkyl, a (C1-C7) alkyl, a (C1-C6) alkyl, a (C1-C5) alkyl, a (C1-C4) alkyl, a (C1-C3) alkyl, a (C1-C2) alkyl, or any combination thereof. In some embodiments, each R is independently selected from the group comprising methyl, ethyl, propyl, butyl, tert-butyl, iso-propyl, hexyl, or a combination thereof. In some embodiments, each R independently comprises a (C1-C8) substituted alkyl, a (C1-C7) substituted alkyl, a (C1-C6) substituted alkyl, a (C1-C5) substituted alkyl, a (C1-C4) substituted alkyl, a (C1-C3) substituted alkyl, a (C1-C2) substituted alkyl, or any combination thereof. In some embodiments, the substituted alkyl comprises one or more substituents, wherein the substituent is as described herein.

In some embodiments, the compound of the invention is or comprises any of:

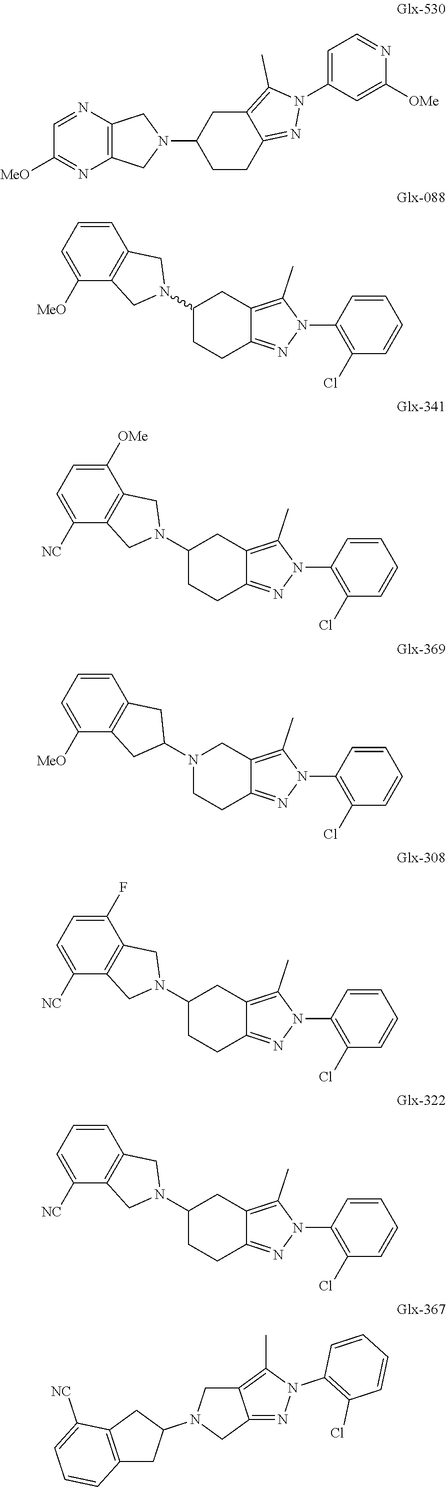

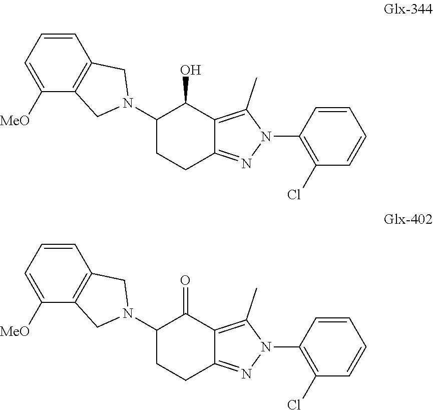 [SMILES: COc1cccc2c1CN(C1CCc3nn(-c4ccccc4Cl)c(C)c3[C@@H]1O)C2 ; COc1cccc2c1CN(C1CCc3nn(-c4ccccc4Cl)c(C)c3C1=O)C2]

In some embodiments, the compound of the invention is any of:

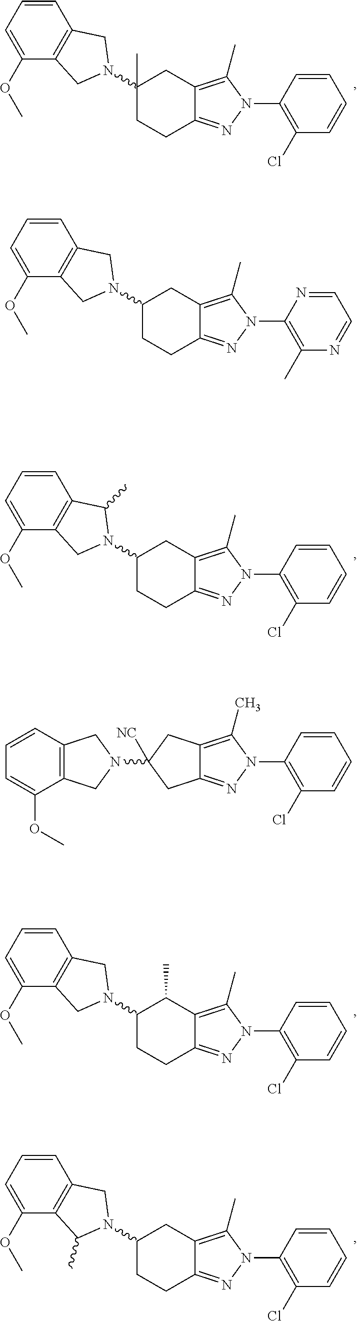

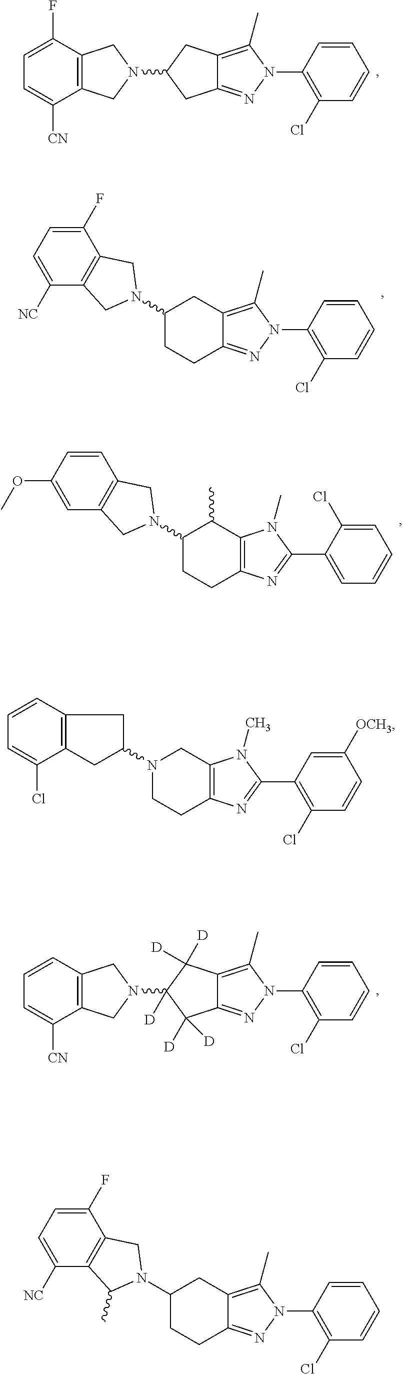

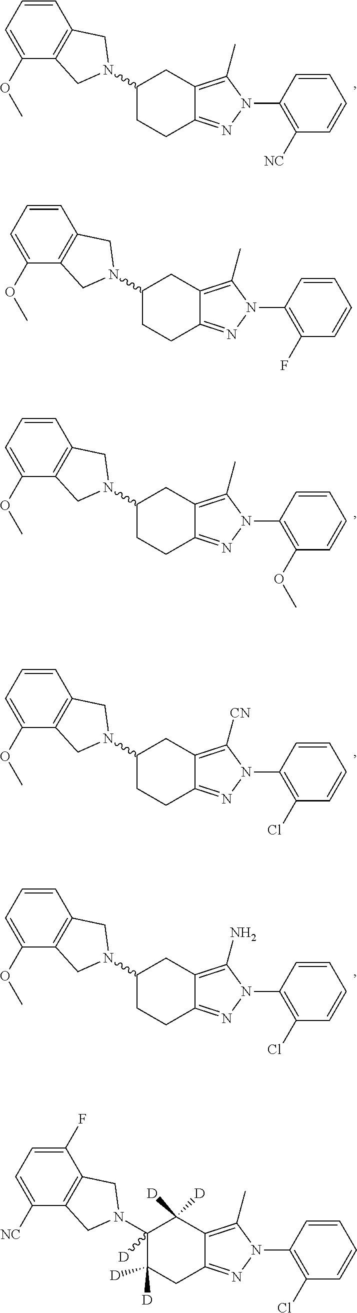

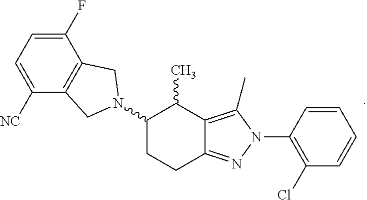 [SMILES: Cc1c2c(nn1-c1ccccc1Cl)CCC(N1Cc3c(F)ccc(C#N)c3C1)C2C]

In some embodiments, the compound of the invention comprises any of the single enantiomers depicted herein or a mixture thereof:

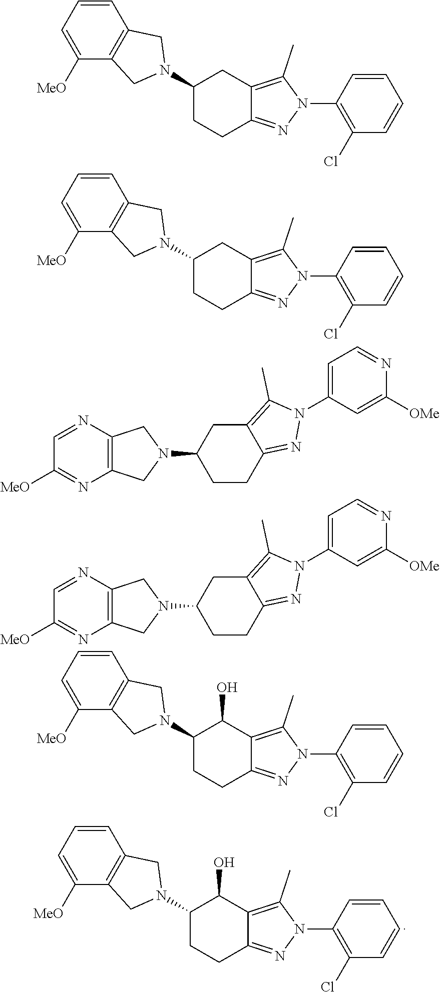

In some embodiments, the compound of the invention substantially comprises a single enantiomer of any one of the compounds described herein, wherein substantially is at least 70%, at least 75%, at least 80%, at least 85%, at least 90%, at least 92%, at least 93%, at least 95%, at least 97%, at least 98%, at least 99% by weight, including any value therebetween.

In some embodiments, the composition of the invention comprises the compound of the invention, a mixture (e.g. racemic mixture) of enantiomers, or is enriched with an enantiomer of interest.

In some embodiments, the compound of the invention comprises a single diastereomer of any one of the compounds described herein, wherein substantially is at least 70%, at least 75%, at least 80%, at least 85%, at least 90%, at least 92%, at least 93%, at least 95%, at least 97%, at least 98%, at least 99% by weight, including any value therebetween. In some embodiments, the composition of the invention comprises a mixture of diastereomers.

In some embodiments, the composition of the invention comprises a mixture of an R-enantiomer and an S-enantiomer, wherein the weight per weight (w/w) ratio between the R-enantiomer and the S-enantiomer within the composition is between 1:10 and 10:1, between 1:1 and 1:3, between 1:3 and 1:5, between 1:5 and 1:10, between 10:1 and 8:1, between 8:1 and 5:1, between 5:1 and 3:1, between 3:1 and 1:1 including any range or value therebetween.

In another aspect, the present invention provides a compound represented by Formula VIIa:

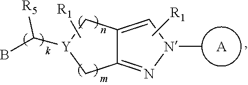

wherein R₁, R₂, R₅, A, B, m, n and Y are as described herein; and wherein k is between 0 and 5. In one embodiment, provided herein is a composition comprising a compound represented by Formula VIIa and a carrier. In one embodiment, provided herein is a pharmaceutically acceptable salt of a compound represented by Formula VIIa.

In some embodiments, A comprises hydrogen, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof. In some embodiments, A comprises an optionally substituted aliphatic (C₃-C₁₀) ring, an optionally substituted aromatic (C₅-C₁₀) ring or a combination thereof. In some embodiments, A comprises an aliphatic (C₃-C₈) ring, an aromatic (C₅ or a C₆) ring, a bicyclic aliphatic (C₅-C₂₀) ring, a bicyclic aromatic (C₆-C₂₀) ring, or a combination thereof, wherein each ring is optionally substituted. In some embodiments, A is devoid of an unsubstituted ring.

In some embodiments, A comprises C₅-C₆ aryl optionally substituted with one or more R₃, C₅-C₆ heteroaryl optionally substituted with one or more R₃, and C₄-C₈ cycloalkyl optionally substituted with one or more R₃ or any combination thereof.

In some embodiments, each R₃ is independently selected from the group comprising hydrogen, halogen, —NO₂, —CN, optionally substituted C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, and optionally substituted C₃-C₈ cycloalkyl or a combination thereof.

In some embodiments, each R is independently selected from the group comprising a (C1-C8) alkyl, a (C3-C8) cycloalkyl, a (C1-C8) substituted alkyl, and hydrogen or is absent. In some embodiments, each R is independently selected from the group comprising a (C1-C5) alkyl, a (C1-C8) substituted alkyl, hydrogen or is absent.

In some embodiments, R comprises a (C1-C8) alkyl, a (C1-C7) alkyl, a (C1-C6) alkyl, a (C1-C5) alkyl, a (C1-C4) alkyl, a (C1-C3) alkyl, a (C1-C2) alkyl, or any combination thereof. In some embodiments, each R is independently selected from the group comprising methyl, ethyl, propyl, butyl, tert-butyl, iso-propyl, hexyl, or a combination thereof.

In some embodiments, R comprises a (C1-C8) substituted alkyl, a (C1-C7) substituted alkyl, a (C1-C6) substituted alkyl, a (C1-C5) substituted alkyl, a (C1-C4) substituted alkyl, a (C1-C3) substituted alkyl, a (C1-C2) substituted alkyl, or any combination thereof. In some embodiments, the substituted alkyl comprises one or more substituents, wherein the substituent is as described herein.

As used herein the term “C₁-C₆ alkyl” including any C₁-C₆ alkyl related compounds, is referred to any linear or branched alkyl chain comprising between 1 and 6, between 1 and 2, between 2 and 3, between 3 and 4, between 4 and 5, between 5 and 6, carbon atoms, including any range therebetween. In some embodiments, C₁-C₆ alkyl comprises any of methyl, ethyl, propyl, butyl, pentyl, iso-pentyl, hexyl, and tert-butyl or any combination thereof. In some embodiments, C₁-C₆ alkyl as described herein further comprises an unsaturated bond, wherein the unsaturated bond is located at 1^{st}, 2^{nd}, 3^{rd}, 4^{th}, 5^{th} or 6^{th} position of the C₁-C₆ alkyl.

As used herein the term “(C₃-C₁₀) ring” is referred to an optionally substituted C3, C4, C5, C6, C7, C8, C9 or C10 ring. In some embodiments, (C₃-C₁₀) ring comprises optionally substituted cyclopropane, cyclobutene, cyclopentane, cyclohexane, or cycloheptane. In some embodiments, (C3-C8) cycloalkyl comprises a cyclic aliphatic compound comprising between 3 and 8, between 3 and 5, between 5 and 6, between 6 and 8, or 3, 4, 5, 6, 7, 8 carbon atoms including any value therebetween, and wherein cyclic aliphatic compound is optionally substituted by one or more substituents, and/or comprises one or more unsaturated bonds.

In some embodiments, R₃ is absent. In some embodiments, R₃ is selected from the group comprising halogen, —NO₂, —CN, —NR₂, —OR, —SR, —NR(C₁-C₆ alkyl), —N(C₁-C₆ alkyl)₂, optionally substituted C₁-C₆ alkyl, wherein R is as described herein. In some embodiments, R is hydrogen. In some embodiments, R₃ is halogen (such as chloro-, or bromo-).

In some embodiments, A comprises any of:

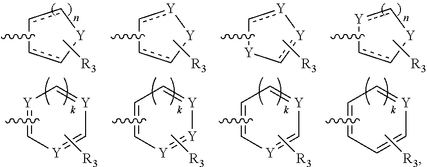

wherein a wavy bond represents an attachment point, wherein k is 0 or 1; wherein n is 1 or 2; and wherein Y and R₃ are as described herein. In some embodiments, A is as described herein wherein each k and n is 1.

In some embodiments, A is a C5 or C6 heteroaryl (e.g. pyrrole, furan, thiophene, thiazole, pyrazole, isothiazole, imidazole, etc.), or of a fused heterocyclic ring (e.g. indole, isoindole, benzofuran, benzothiophene, benzotriazole, quinoline, chromene, chroman, quinazoline), optionally substituted with one or more R₃. In some embodiments, A is a phenyl optionally substituted with one or more R₃. In some embodiments, A is as described herein wherein each Y is independently selected from —N—, —NH, and —NR, wherein R is as described herein. In some embodiments, A is attached via a carbon atom. In some embodiments, A is attached via a nitrogen atom.

In some embodiments, R₃ is attached at any of the ortho-, meta- or para-positions. In some embodiments, A is phenyl optionally substituted with one or more R₃ at ortho position. In some embodiments, R₃ is attached at any of the ortho-, meta- or para-positions. In some embodiments, A comprises phenyl optionally substituted with one or more halogen at ortho position. As used herein the ortho-, meta- or para-positions are with respect to the attachment point of A. In some embodiments, A comprises phenyl substituted by a halogen. In some embodiments, A comprises phenyl substituted by one or more C₁-C₆ alkyl.

In some embodiments, A comprises an alkaryl (such as benzyl) optionally substituted with one or more R₃, wherein R₃ is as described herein.

In some embodiments, B comprises bicyclic aryl optionally substituted with one or more R₄. In some embodiments, B comprises bicyclic heteroaryl optionally substituted with one or more R₄. In some embodiments, B comprises bicyclic heterocyclyl optionally substituted with one or more R₄. In some embodiments, B comprises bicyclic cycloalkyl optionally substituted with one or more R₄.

In some embodiments, each R₄ is independently selected from the group comprising hydrogen, halogen, —NO₂, —CN, C₁-C₆ alkyl optionally substituted with one or more R₅, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, optionally substituted C₃-C₈ cycloalkyl, optionally substituted heterocyclyl, optionally substituted aryloxy, optionally substituted heteroaryloxy, optionally substituted cycloalkyloxy, 2-hydroxy-3-methoxypropoxy, (2-methoxyethoxy)methyl, and 2-(3-(but-3-yn-1-yl)-3H-diazirin-3-yl)ethoxy or a combination thereof.

In some embodiments, R₄ is attached to a carbon atom or to a heteroatom. In some embodiments, R₄ attached to a carbon atom comprises hydrogen, halogen, —NO₂, —CN, C₁-C₆ alkyl optionally substituted with one or more R₅, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, optionally substituted C₃-C₈ cycloalkyl, optionally substituted heterocyclyl, optionally substituted aryloxy, optionally substituted heteroaryloxy, optionally substituted cycloalkyloxy, 2-hydroxy-3-methoxypropoxy, (2-methoxyethoxy)methyl, and 2-(3-(but-3-yn-1-yl)-3H-diazirin-3-yl)ethoxy or a combination thereof.

In some embodiments, R₄ attached to a carbon atom comprises hydrogen, halogen (e.g. Cl—, Br—, F—), —NO₂, —CN, C₁-C₆ alkyl optionally substituted with one or more R₅, —NH₂, —NH(C₁-C₆ alkyl), —N(C₁-C₆ alkyl)₂, —OH, or a combination thereof.

In some embodiments, R₄ attached to a heteroatom comprises hydrogen, C₁-C₆ alkyl optionally substituted with one or more R₅, C₁-C₆ haloalkyl, C₁-C₆ alkoxy, C₁-C₆ haloalkoxy, alkoxy(C₁-C₆ alkyl), alkoxy(C₁-C₆ alkoxy), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, optionally substituted C₃-C₈ cycloalkyl, optionally substituted heterocyclyl, optionally substituted aryloxy, optionally substituted heteroaryloxy, optionally substituted cycloalkyloxy, 2-hydroxy-3-methoxypropoxy, (2-methoxyethoxy)methyl, and 2-(3-(but-3-yn-1-yl)-3H-diazirin-3-yl)ethoxy or a combination thereof. In some embodiments, R₄ attached to a heteroatom comprises hydrogen, C₁-C₆ alkyl optionally substituted with one or more R₅, or both.

In some embodiments, R₅ comprises hydrogen, deuterium, halogen, hydroxy, amino, C₁-C₆ alkyl, C₁-C₆ haloalkyl, optionally substituted C₃-C₈ cycloalkyl, —CO₂R, —CN, —CONRR, or a combination thereof, wherein R is as described herein. In some embodiments, R₅ comprises hydrogen. In some embodiments, the compound of the invention is a deuterated, wherein one or two R₅ comprise deuterium.

In some embodiments, B comprises 7-10 bicyclic ring optionally substituted with one or more R₃, wherein the bicyclic ring optionally comprises 1 or 2 heteroatoms.

In some embodiments, B comprises a fused 7-10 aliphatic ring optionally substituted with one or more R₄. In some embodiments, fused 7-10 aliphatic ring comprises norbornane, bicyclooctane, bicyclodecane or a combination thereof. In some embodiments, at least one ring of the bicyclic ring comprises a heterocyclic aliphatic ring. In some embodiments, the heterocyclic aliphatic ring comprises oxirane, tetrahydrofuran, aziridine, pyrrolidine, piperidine, or morpholine.

As used herein, the term “7-10 ring” is referred to a cyclic aliphatic or aromatic compound comprising between 7 and 10 carbon atoms. In some embodiments, 7-10 ring bicyclic ring comprises between 7 and 8, between 8 and 9, between 9 and 10 carbon atoms including any value therebetween.

In some embodiments, B comprises a fused 7-10 heterocyclic ring optionally substituted with one or more R₄, wherein fused 7-10 heterocyclic ring is as described herein.

In some embodiments, B comprises any of:

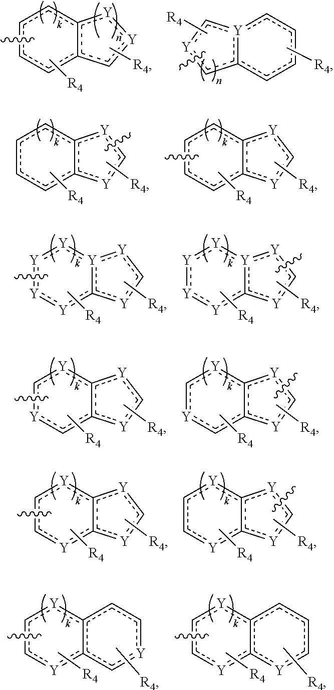

wherein Y and R₄ are as described hereinabove and wherein at least one Y is a heteroatom. In some embodiments, at least two Y independently represent heteroatom.

In some embodiments, each Y is independently selected from the group comprising —CH—, —CR—, —CH₂—, —NH—, —NR—, —CR₂—, —S—, —O—, and —N—. In some embodiments, Y is —NH— or —N—. In some embodiments, Y is —CH— or —C—.

In another aspect of the invention, the compound of the invention is represented by Formula VIIb: or by Formula VIIc:

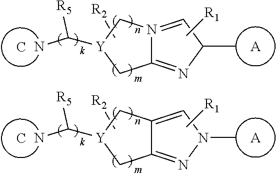

wherein R₁, R₂, A, B, m, n and Y are as described herein; wherein k is between 0 and 5; and wherein C comprises optionally substituted C₃-C₈ heterocyclyl, optionally substituted heteroaryl, optionally substituted bicyclic heteroaryl, optionally substituted bicyclic heterocyclyl, or a combination thereof.

In some embodiments, C comprises any of:

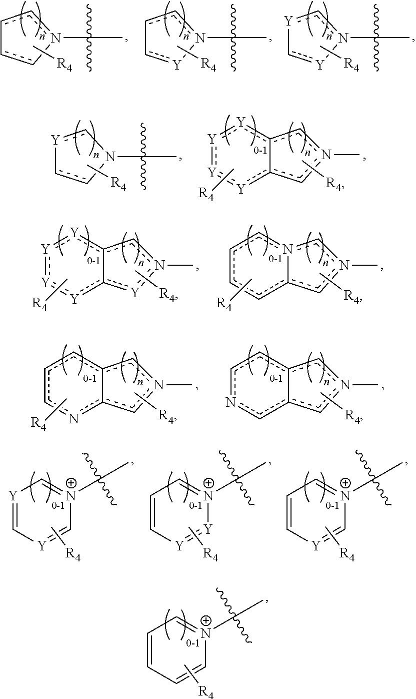

wherein Y, R₄, and n are as described herein.

In some embodiments, C comprises optionally substituted bicyclic C9 or C10 heterocyclyl. In some embodiments, C9 or C10 heterocyclyl comprises benzothiazole, benzoxazole, benzotriazole, indole, isoindole, benzofuran, isobenzofuran, 1-benzothiophene, 2-benzothiophene, indazole, purine, or a combination thereof.

In another aspect of the invention, the compound of the invention is represented by or comprises Formula IIa: or by Formula IIb:

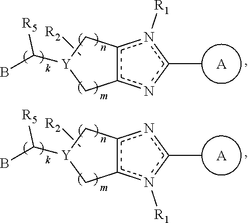

wherein R₁, R₂, R₅, A and Y are as described herein; wherein if Y is N, then k is 1; and if Y is —C— or —CH—, then k is between 0 and 5.

In some embodiments, if k is 1 then B represents a fused 7-10 ring comprising at least one heteroatom and optionally substituted with one or more R₄. In some embodiments, if k is 1 then B represents a fused 7-10 aliphatic ring comprising at least one heteroatom and optionally substituted with one or more R₄. In some embodiments, if k is 1 then B represents a fused 7-10 at least partially unsaturated ring comprising at least one heteroatom and optionally substituted with one or more R₄. In some embodiments, if k is 1 then B represents a fused 7-10 aromatic ring comprising at least one heteroatom and optionally substituted with one or more R₄.

In some embodiments, if k is 1 then the fused 7-10 aromatic ring comprising at least one heteroatom is devoid of indole. In some embodiments, if k is 1 then the fused 7-10 aromatic ring comprising at least one heteroatom comprises indole attached to methylene at position 2. In some embodiments, if k is 1 then B is devoid of indole attached to methylene at position 3. In some embodiments, if k is 1 then B is devoid of carbazole. In some embodiments, if k is 1 then B is devoid of quinoline. In some embodiments, if k is 0, then B is devoid of 2,3-dihydrobenzofuran. In some embodiments, if k is 1 then B is devoid of 1,3-benzodioxole. In some embodiments, if k is 1 then B is devoid of chromen-4-one.

In some embodiments, B comprises any of:

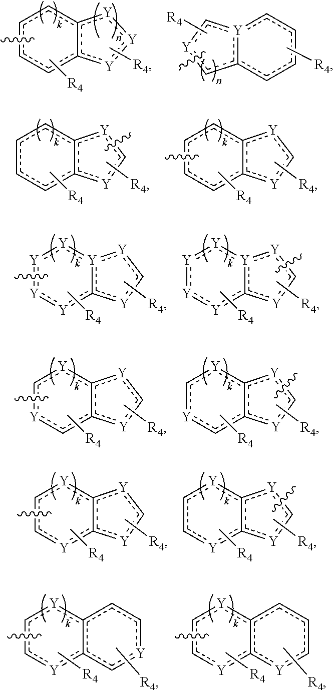

wherein Y and R₄ are as described hereinabove and wherein at least one Y is a heteroatom. In some embodiments, at least two Y independently represent heteroatoms.

In some embodiments, Y is —C— or —CH— and k is between 0 and 1, between 1 and 2, between 2 and 3, between 3 and 4, between 4 and 5 including any range or value therebetween. In some embodiments, Y is —C— or —CH— and k is 1 or 2. In some embodiments, Y is —C— or —CH— and k is 0.

In some embodiments, R₁ represents a substituent independently comprising hydrogen, optionally substituted C₁-C₆ alkyl, C₁-C₆ haloalkyl, C₁-C₆ alkoxy, C₁-C₆ haloalkoxy, hydroxy(C₁-C₆ alkyl), 3-oxetanyl, 2-optionally substituted aryl, optionally substituted heteroaryl, optionally substituted heterocyclyl, and optionally substituted C₃-C₈ cycloalkyl or a combination thereof, or is absent.

In some embodiments, R₁ represents a substituent selected from the group comprising optionally substituted C₁-C₆ alkyl, C₁-C₆ haloalkyl, C₁-C₆ alkoxy, C₁-C₆ haloalkoxy, hydroxy(C₁-C₆ alkyl), 3-oxetanyl, or a combination thereof, wherein substituted alkyl comprises a substituent selected from halogen (e.g. Cl—, Br—, F—), —NO₂, —CN, a carboxylic acid derivative, C₁-C₆ alkyl, —NH₂, —NH(C₁-C₆ alkyl), —N(C₁-C₆ alkyl)₂, —OH, or a combination thereof.

In some embodiments, R₁ comprises hydrogen. In some embodiments, R₁ comprises halogen. In some embodiments, R₁ is bound to a heteroatom (such as nitrogen). In some embodiments, R₁ comprises C₁-C₆ alkyl. In some embodiments, R₁ comprises hydrogen, or optionally substituted C₁-C₄ alkyl. In some embodiments, R₁ comprises a (C1-C8) alkyl, a (C1-C7) alkyl, a (C1-C6) alkyl, a (C1-C5) alkyl, a (C1-C4) alkyl, a (C1-C3) alkyl, a (C1-C2) alkyl, or any combination thereof. In some embodiments, R₁ comprises a (C1-C8) substituted alkyl, a (C1-C7) alkyl, a (C1-C6) substituted alkyl, a (C1-C5) substituted alkyl, a (C1-C4) substituted alkyl, a (C1-C3) substituted alkyl, a (C1-C2) substituted alkyl, or any combination thereof; wherein the substituted alkyl is as described herein. In some embodiments, R₁ is absent.

In some embodiments, each R₂ is independently selected from the group comprising halogen, —NO₂, —CN, optionally substituted C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂ or a combination thereof.

In some embodiments, each R₂ is independently selected from the group comprising a (C1-C6) alkyl, halo, a (C1-C6) alkoxy, hydroxy, amino, a (C1-C6) thioalkoxy, mercapto, cyano, a carboxylic acid derivative, nitro, guanidine, a heteroatom, and hydrogen or any combination thereof.

In some embodiments, R₂ is absent. In some embodiments, each R₂ is independently selected from the group comprising a (C1-C5) alkyl, halo, a (C1-C5) alkoxy, a (C1-C4) alkoxy, a (C1-C3) alkoxy, a (C1-C2) alkoxy, hydroxy, amino, a (C1-C5) thioalkoxy, a (C1-C4) thioalkoxy, a (C1-C3) thioalkoxy, a (C1-C2) thioalkoxy, mercapto, cyano, a carboxylic acid derivative, nitro, guanidine, a heteroatom, and hydrogen or any combination thereof, or is absent. In some embodiments, R₂ comprises a (C1-C8) alkyl, a (C1-C7) alkyl, a (C1-C6) alkyl, a (C1-C5) alkyl, a (C1-C4) alkyl, a (C1-C3) alkyl, a (C1-C2) alkyl, or any combination thereof. In some embodiments, 10 comprises a (C1-C8) substituted alkyl, a (C1-C7) alkyl, a (C1-C6) substituted alkyl, a (C1-C5) substituted alkyl, a (C1-C4) substituted alkyl, a (C1-C3) substituted alkyl, a (C1-C2) substituted alkyl, or any combination thereof, wherein the substituted alkyl is as described herein.

In some embodiments, each R₁ and R₂ is independently selected from the group comprising methyl, ethyl, propyl, butyl, tert-butyl, iso-propyl, hexyl, or a combination thereof.

In some embodiments, the compound of the invention is represented by or comprises Formula Vb: or Formula Vb1:

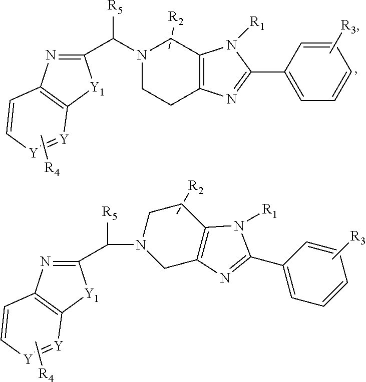

wherein R₁, R₂, R₃, R₄, R₅, and Y are as described herein and Y₁ is selected from the group consisting of N, NH, S, and O. In some embodiments, the compound of Formula Va or Vb, wherein Y comprises nitrogen.

In some embodiments, the compound of the invention comprises any of compounds 1 to 15:

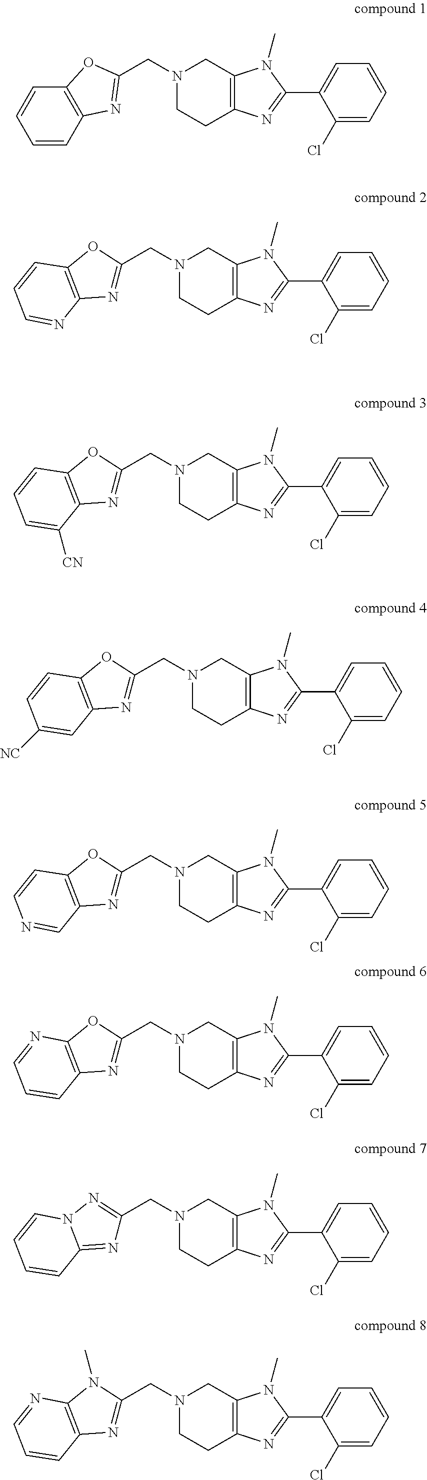

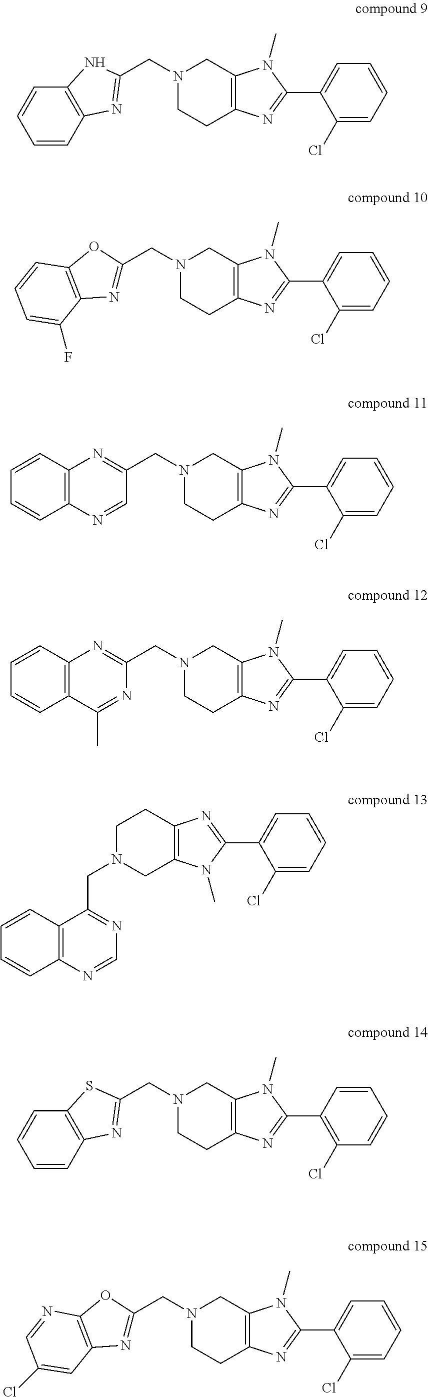

In some embodiments, the compound of the invention comprises any of:

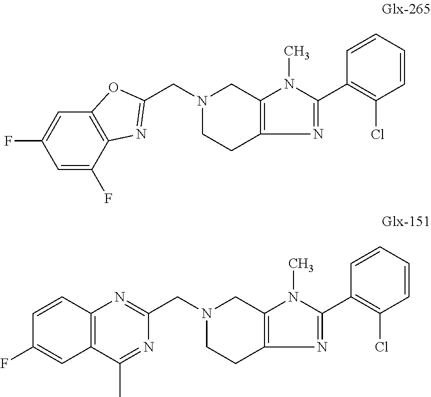 [SMILES: Cn1c(-c2ccccc2Cl)nc2c1CN(Cc1nc3c(F)cc(F)cc3o1)CC2 ; Cc1nc(CN2CCc3nc(-c4ccccc4Cl)n(C)c3C2)nc2ccc(F)cc12]

In some embodiments, the compound of the invention comprises any one of the compounds disclosed herein, including any enantiomers thereof. In some embodiments, the compound of the invention comprises a mixture of enantiomers (e.g. a racemic mixture).

### Pharmaceutical Composition

In another aspect of the invention disclosed herein, there is a pharmaceutical composition comprising the compound of the invention, a pharmaceutically acceptable salt thereof or both.

Non-limiting examples of pharmaceutically acceptable salts include but are not limited to: acetate, aspartate, benzenesulfonate, benzoate, bicarbonate, carbonate, halide (such as bromide, chloride, iodide, fluoride), bitartrate, citrate, salicylate, stearate, succinate, sulfate, tartrate, decanoate, edetate, fumarate, gluconate, and lactate or any combination thereof.

In some embodiments, the pharmaceutical composition comprises the compound of the invention and a pharmaceutically acceptable carrier. In some embodiments, the pharmaceutical composition comprises a therapeutically effective amount of the compound of the invention and the pharmaceutically acceptable carrier.

For example, the term “pharmaceutically acceptable” can mean approved by a regulatory agency of the Federal or a state government or listed in the U.S. Pharmacopeia or other generally recognized pharmacopeia for use in animals, and more particularly in humans. In some embodiments, the compound of the invention is referred to herein as an active ingredient of a pharmaceutical composition.

In some embodiments, the pharmaceutical composition as described herein is a topical composition. In some embodiments, the pharmaceutical composition is an oral composition. In some embodiments, the pharmaceutical composition is an injectable composition. In some embodiments, the pharmaceutical composition is for a systemic use.

In some embodiments, the pharmaceutical composition is any of an emulsion, a liquid solution, a gel, a paste, a suspension, a dispersion, an ointment, a cream or a foam.

As used herein, the term “carrier” refers to a diluent, adjuvant, excipient, or vehicle with which the active ingredient is administered. Such carriers can be sterile liquids, such as water-based and oils, including those of petroleum, animal, vegetable or synthetic origin, such as peanut oil, soybean oil, mineral oil, sesame oil and the like, polyethylene glycols, glycerin, propylene glycol or other synthetic solvents.

Other non-limiting examples of carriers include, but are not limited to: terpenes derived from Cannabis, or total terpene extract from Cannabis plants, terpenes from coffee or cocoa, mint-extract, eucalyptus-extract, citrus-extract, tobacco-extract, anis-extract, any vegetable oil, peppermint oil, d-limonene, b-myrcene, a-pinene, linalool, anethole, a-bisabolol, camphor, b-caryophyllene and caryophyllene oxide, 1,8-cineole, citral, citronella, delta-3-carene, farnesol, geraniol, indomethacin, isopulegol, linalool, unalyl acetate, b-myrcene, myrcenol, 1-menthol, menthone, menthol and neomenthol, oridonin, a-pinene, diclofenac, nepafenac, bromfenac, phytol, terpineol, terpinen-4-ol, thymol, and thymoquinone. One skilled in the art will appreciate, that a particular carrier used within the pharmaceutical composition of the invention may vary depending on the route of administration.

In some embodiments, the carrier improves the stability of the active ingredient in a living organism. In some embodiments, the carrier improves the stability of the active ingredient within the pharmaceutical composition. In some embodiments, the carrier enhances the bioavailability of the active ingredient.

Water may be used as a carrier such as when the active ingredient has a sufficient aqueous solubility, so as to be administered intravenously. Saline solutions and aqueous dextrose and glycerol solutions can also be employed as liquid carriers, particularly for injectable solutions.

In some embodiments, the carrier is a liquid carrier. In some embodiments, the carrier is an aqueous carrier.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from 0.1% to 99.99999% by weight of the composition/s or the pharmaceutical composition/s presented herein.

In some embodiments, the pharmaceutical composition includes incorporation of any one of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions may influence the physical state, solubility, stability, rate of in vivo release, and rate of in vivo clearance.

In some embodiments, the pharmaceutical composition is a liquid at a temperature between 15 to 45° C. In some embodiments, the pharmaceutical composition is a solid at a temperature between 15 to 45° C. In some embodiments, the pharmaceutical composition is a semi-liquid at a temperature between 15 to 45° C. It should be understood that the term “semi-liquid”, is intended to mean materials which are flowable under pressure and/or shear force. In some embodiments, semi-liquid compositions include creams, ointments, gel-like materials and other similar materials. In some embodiments, the pharmaceutical composition is a semi-liquid composition, characterized by a viscosity in a range from 31,000-800,000 cps.

Non-limiting examples of carriers for pharmaceutical compositions being in the form of a cream include but are not limited to: non-ionic surfactants (e.g., glyceryl monolinoleate glyceryl monooleate, glyceryl monostearate lanolin alcohols, lecithin mono- and di-glycerides poloxamer polyoxyethylene 50 stearate, and sorbitan trioleate stearic acid), anionic surfactants (e.g. pharmaceutically acceptable salts of fatty acids such as stearic, oleic, palmitic, and lauric acids), cationic surfactants (e.g. pharmaceutically acceptable quaternary ammonium salts such as benzalkonium chloride, benzethonium chloride, and cetylpyridinium chloride) or any combination thereof.

In some embodiments, the pharmaceutical composition being in the form of a cream further comprises a thickener.

Non-limiting examples of thickeners include, but are not limited to microcrystalline cellulose, a starch, a modified starch, gum tragacanth, gelatin, and a polymeric thickener (e.g. polyvinylpyrrolidone) or any combination thereof.

In some embodiments, the pharmaceutical composition comprising the compound of the invention is in a unit dosage form. In some embodiments, the pharmaceutical composition is prepared by any of the methods well known in the art of pharmacy. In some embodiments, the unit dosage form is in the form of a tablet, capsule, lozenge, wafer, patch, ampoule, vial or pre-filled syringe.

In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems. In some embodiments, the effective dose is determined as described hereinabove.

In another embodiment, the pharmaceutical composition of the invention is administered in any conventional oral, parenteral or transdermal dosage form.

As used herein, the terms “administering”, “administration”, and like terms refer to any method which, in sound medical practice, delivers a composition containing an active agent to a subject in such a manner as to provide a therapeutic effect.

In some embodiments, the pharmaceutical composition is administered via oral (i.e., enteral), rectal, vaginal, topical, nasal, ophthalmic, transdermal, subcutaneous, intramuscular, intraperitoneal or intravenous routes of administration. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. In addition, it may be desirable to introduce the pharmaceutical composition of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

In some embodiments, the pharmaceutical composition is in a form of, for example, and not by way of limitation, an ointment, cream, gel, paste, foam, aerosol, suppository, pad or gelled stick.

In some embodiments, for oral applications, the pharmaceutical composition or is in the form of a tablets or a capsule, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. In some embodiments, the tablet of the invention is further film coated. In some embodiments, oral application of the pharmaceutical composition or of the kit is in a form of a drinkable liquid. In some embodiments, oral application of the pharmaceutical composition or of the kit is in a form of an edible product.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

In some embodiments, the pharmaceutical composition is for use in the inhibition of glioma-associated oncogene (Gli). In some embodiments, inhibition of Gli comprises inhibition of cellular activity of Gli, wherein inhibition is as described herein. In some embodiments, Gli is Gli 1. In some embodiments, inhibition of Gli comprises a selective inhibition of Gli1 activity.

In some embodiments, the compound has at least 5 times, at least 10 times, at least 15 times, at least 20 times, at least 30 times, at least 30 times, at least 50 times, at least 80 times, at least 100 times, at least 200 times, at least 300 times, at least 400 times, at least 500 times, at least 700 times, at least 1000 times, at least 10,000 times, at least 50,000 times, at least 100,000 times lower minimum inhibitory concentration (MIC) for the Gli1 as compared to other Gli proteins (e.g. Gli 2 or Gli 3).

In some embodiments, the compound of the invention significantly inhibits Gli1 activity (as represented in the Examples section). In some embodiments, compounds 1 to 5 and 7 to 17 inhibit 50% of Gli1 activity at a concentration of less than 5000 nM, less than 2000 nM, less than 1000 nM, less than 200 nM, less than 500 nM, less than 50 nM, less than 10 nM, less than 5 nM, less than 1 nM. In some embodiments, inhibition of Gli1 activity is evaluated in-vitro (as represented in the Examples section). In some embodiments, compounds 11, 13, 14, inhibit 50% of Gli1 activity at a concentration of less than 17 nM. According to the experimental results some of the pyrazole-based compounds and inhibit 50% of Gli1 activity at a concentration of less than 1 nM.

In some embodiments, the pharmaceutical composition is for use in the prevention or inhibition of Gli associated cell toxicity. In some embodiments, the pharmaceutical composition is for use in the prevention or inhibition of demyelination.

In some embodiments, the cell is a cancerous cell. In some embodiments, the cell is expresses a Gli protein. In some embodiments, the cell is characterized by an elevated expression of Gli 1. In some embodiments, the cell is characterized by an elevated Gli 1 expression. In some embodiments, the cell is a neuron.

In some embodiments, the cell is selected from the group comprising a breast cancer cell, a pancreatic cancer cell, a colon cancer cell, a lung cancer cell, rhabdomyosarcoma cell, a basal-cell carcinoma cell, a glioblastoma cell, a medulloblastoma cell, a tumor cell, a solid tumor cell, an esophageal cancer cell, a leukemia cell, a prostate cancer cell, an osteosarcoma cell, a liver cancer cell, a thyroid cancer cell, an ovarian cancer cell, a skin cancer cell, a mesothelioma cell and a lymphoma cancer cell, or any combination thereof.

In some embodiments, the pharmaceutical composition is for use in the prevention or treatment of abnormal cell growth of a cell expressing Gli 1 in a subject in need thereof. In some embodiments, the pharmaceutical composition is for enhancing remyelination in a subject in need thereof.

In some embodiments, the pharmaceutical composition is for use in the prevention or treatment of abnormal proliferation of cells expressing Gli 1.

### Method

In another aspect, there provided herein is a method for preventing, ameliorating and/or treating a neurological disorder comprising administering to the subject the pharmaceutical composition or the compound of the invention. In some embodiments, there is a method is for preventing or treating cancer comprising administering to the subject the pharmaceutical composition or the compound of the invention. In some embodiments, administering is by an oral administration, a topical administration, a systemic administration or a combination thereof.

In some embodiments, the method is for preventing or treating cancer characterized by a cell expressing Gli. In some embodiments, Gli is Gli1. In some embodiments, the method is for prevention or inhibition of cell mitosis, wherein the cell is as described hereinabove.

In some embodiments, the method comprises administering a compound as described herein to a subject afflicted with cancer. In some embodiments, the method comprises administering a compound as described herein to a subject afflicted with cancer characterized by cancerous cells that express Gli and/or highly express Gli. In some embodiments, the method further comprises selecting a subject afflicted with cancer wherein and comprising a cancerous cell expressing Gli and/or highly expressing Gli.

In some embodiments, the neurological disorder to be treated with a compound and/or a composition as described herein, is selected from: multiple sclerosis, central pontine myelinolysis, acute disseminated encephalomyelitis, progressive multifocal leukoencephalopathy, subacute sclerosing panencephalitis, post-infectious encephalomyelitis, chronic inflammatory demyelinating polyneuropathy, Devic's disease, Balo's concentric sclerosis, the leukodystrophies, optic neuritis, transverse myelitis, cerebral palsy, spinal cord injury, age-associated myelin deficiency, Alzheimer's Disease, and acquired and inherited neuropathies in the peripheral nervous system. In some embodiments, the neurological disorder is multiple sclerosis. In some embodiments, the neurological disorder is Alzheimer's Disease.

In some embodiments, the method is for enhancing remyelination, the method comprising administering one or more compounds of the invention or the pharmaceutical composition of the invention to a subject in need of remyelination enhancement therapy.

As used herein, the term “remyelination” refers to the generation of new myelin sheaths around axons. This follows the pathological loss of myelin in a neurological disorder. Remyelination can restore conduction properties to axons (thereby restoring neurological function) and is increasingly believed to exert a neuroprotective role on axons.

Remyelination and identification of a subject that can benefit from a compound and/or a composition as describe herein, can be monitored/identified by methods which include determination of the state of myelin in the subject, such as by Mill, PET, PET-CT, etc. Treatment effectiveness can be evaluated by monitoring physiological parameters of the subject. Such physiological parameters are well-known in the art.

In some embodiments, the method comprises administering the pharmaceutical composition of the invention at least 1 time, at least 2 times, at least 3 times, at least 4 times, at least 5 times, at least 7 times, or at least 10 times per day or per week or per month, or any value and range therebetween. Each possibility represents a separate embodiment of the invention. In some embodiments, the method comprises administering the composition or the combination of the invention 1-2 times per day or per week or per month, 1-3 times per day or per week or per month, 1-4 times per day or per week or per month, 1-5 times per day, 1-7 times per day or per week or per month, 2-3 times per day or per week or per month, 2-4 times per day or per week or per month, 2-5 times per day or per week or per month, 3-4 times per day or per week or per month, 3-5 times per day or per week or per month, or 5-7 times per day or per week or per month. Each possibility represents a separate embodiment of the invention.

In some embodiments, the method comprises administering the pharmaceutical composition of the invention to the subject at a daily or weekly or monthly dosage of 0.05 to 20 mg/kg, 0.05 to 0.1 mg/kg, 0.1 to 0.3 mg/kg, 0.3 to 0.5 mg/kg, 0.5 to 0.8 mg/kg, 0.8 to 1 mg/kg, 1 to 2 mg/kg, 2 to 5 mg/kg, 5 to 10 mg/kg, 10 to 15 mg/kg, 15 to 20 mg/kg including any range or value therebetween.

It should be apparent to one skilled in the art, that in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

In some embodiments, the subject is a mammal. In some embodiments, the subject is a lab animal. In some embodiments, the subject is a pet. In some embodiments, the subject is a rodent. In some embodiments, the subject is a farm animal. In some embodiments, the subject is a human subject.

In some embodiments, the composition of the present invention is administered in a therapeutically safe and effective amount. As used herein, the term “safe and effective amount” refers to the quantity of a component which is sufficient to yield a desired therapeutic response without undue adverse side effects, including but not limited to toxicity, such as calcemic toxicity, irritation, or allergic response, commensurate with a reasonable benefit/risk ratio when used in the presently described manner. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005). In some embodiments, the effective amount or dose of the active ingredient can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages may vary depending on the dosage form employed and the route of administration utilized. In one embodiment, the exact formulation, route of administration and dosage can be chosen by the individual physician in view of the patient's condition. [See e.g., Goodman and Gilman's The Pharmacological Basis of Therapeutics, 13th Ed., McGraw-Hill/Education, New York, N.Y. (2017)].

In some embodiments, the subject is afflicted with a disease or disorder associated with an abnormal Gli1 expression. In some embodiments, the subject is afflicted with a disease or disorder selected from the group comprising: breast cancer, pancreatic cancer, colon cancer, lung cancer, rhabdomyosarcoma, basal-cell carcinoma, glioblastoma, medulloblastoma, leukemia, prostate cancer, skin cancer, lymphoma, esophageal cancer, ovarian cancer, thyroid cancer, osteosarcoma, liver cancer, multiple endocrine neoplasia, gastrointestinal cancer, or mesothelioma.

In some embodiments, the subject is afflicted with a neurological disease or disorder selected from the group comprising: multiple sclerosis, central pontine myelinolysis, acute disseminated encephalomyelitis, progressive multifocal leukoencephalopathy, subacute sclerosing panencephalitis, post-infectious encephalomyelitis, chronic inflammatory demyelinating polyneuropathy, Devic's disease, Balo's concentric sclerosis, leukodystrophy, optic neuritis, transverse myelitis, cerebral palsy, spinal cord injury, age-associated myelin deficiency, Alzheimer's Disease, and acquired and inherited neuropathy in the peripheral nervous system or any combination thereof.

In some embodiments, the method is for reducing or inhibiting: abnormal cell proliferation, tumor growth, malignancy, or any combination thereof in a subject in need thereof. In some embodiments, the method is for reducing or inhibiting proliferation of cells expressing Gli. In some embodiments, the method is for selectively reducing or inhibiting proliferation of cells expressing Gli1. In some embodiments, the method is for reducing or inhibiting the activity of Gli1, wherein the Gli1 activity comprises activation of gene transcription. In some embodiments, the method is used in a subject having a disease wherein a diseased cell expresses Gli1.

In some embodiments, reducing comprises at least 50%, at least 60%, at least 70%, at least 80%, at least 85%, at least 90%, at least 92%, at least 95%, at least 97%, at least 99% reduction of the cell proliferation, including any value therebetween.

In some embodiments, reducing comprises at least 50%, at least 60%, at least 70%, at least 80%, at least 85%, at least 90%, at least 92%, at least 95%, at least 97%, at least 99% reduction of Gli1 activity, including any value therebetween.

In some embodiments, the compound of the invention has IC₅₀ in inhibiting Gli1 activity between 0.1 and 1 nM, between 1 and 5 nM, between 5 and 10 nM, between 10 and 50 nM, between 50 and 100 nM, between 100 and 500 nM, between 500 and 1 uM, between 1 and 5 uM, between 5 and 10 uM, including any value therebetween.

In some embodiments, the compound of the invention is substantially inactive with respect to the cells expressing other Gli proteins (such as Gli2 and Gli3). In some embodiments, the method is for selectively inhibiting the activity of Gli1. In some embodiments, the compound of the invention has an increased affinity to the Gli1 mutant as compared to other Gli proteins.

In some embodiments, the compound has at least 5 times, at least 10 times, at least 15 times, at least 20 times, at least 30 times, at least 30 times, at least 50 times, at least 80 times, at least 100 times, at least 200 times, at least 300 times, at least 400 times, at least 500 times, at least 700 times, at least 1000 times, at least 10,000 times, at least 50,000 times, at least 100,000 times lower minimum inhibitory concentration (MIC) for Gli1 as compared to other Gli proteins.

### Definitions

As used herein, the term “alkyl” describes an aliphatic hydrocarbon including straight chain and branched chain groups. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. In some embodiments, the term “substituted” or the term “substituent” is referred to 1, 2, 3, 4 or 5 substituents, wherein each substituent is independently selected from (C₀-C₆)alkyl-aryl, (C₀-C₆)alkyl-heteroaryl, (C₀-C₆)alkyl-(C₃-C₈) cycloalkyl, optionally substituted C₃-C₈ heterocyclyl, halogen, NO₂, CN, OH, CONH₂, CONR₂, CNNR₂, CSNR₂, CONH—OH, CONH—NH₂, NHCOR, NHCSR, NHCNR, —NC(═O) OR, —NC(═O)NR, —NC(═S) OR, —NC(═S)NR, SO₂R, SOR, —SR, SO₂OR, SO₂N(R)₂, —NHNR₂, —NNR, C₁-C₆ haloalkyl, optionally substituted C₁-C₆ alkyl, NH₂, NH(C₁-C₆ alkyl), N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkylNR₂, C₁—C₆ alkylSR, CONH(C₁-C₆ alkyl), CON(C₁-C₆ alkyl)₂, CO₂H, CO₂R, —OCOR, —OCOR, —OC(═O)OR, —OC(═O)NR, —OC(═S)OR, —OC(═S)NR, including nay combination thereof.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein. or “carboxylate”

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)₂R′ group, where R′ is as defined herein.

A “carbamyl” or “carbamate” group describes an —OC(O)NR′R″ group, where R′ is as defined herein and R″ is as defined for R′. A “nitro” group refers to a —NO₂ group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)— linking group, as these phrases are defined hereinabove, where R′ and R″ are as defined herein.

The term “carboxylic acid derivative” as used herein encompasses carboxy, amide, carbonyl, anhydride, carbonate ester, and carbamate. A “cyano” or “nitrile” group refers to a —CN group. The term “azo” or “diazo” describes an —N═NR′ end group or an —N═N— linking group, as these phrases are defined hereinabove, with R′ as defined hereinabove. The term “guanidine” describes a —R′NC(N)NR″R′″ end group or a —R′NC(N) NR″— linking group, as these phrases are defined hereinabove, where R′, R″ and R″ are as defined herein. As used herein, the term “azide” refers to a —N₃ group. The term “sulfonamide” refers to a —S(O)₂NR′R″ group, with R′ and R″ as defined herein.

The term “phosphonyl” or “phosphonate” describes an —OP(O)—(OR′)₂ group, with R′ as defined hereinabove. The term “phosphinyl” describes a —PR′R″ group, with R′ and R″ as defined hereinabove. The term “alkylaryl” describes an alkyl, as defined herein, which substituted by an aryl, as described herein. An exemplary alkylaryl is benzyl.

The term “heteroaryl” describes a monocyclic or fused ring (i.e. rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazol, pyridine, pyrrole, oxazole, indole, purine and the like.

As used herein, the terms “halo” and “halide”, which are referred to herein interchangeably, describe an atom of a halogen, that is fluorine, chlorine, bromine or iodine, also referred to herein as fluoride, chloride, bromide and iodide.

### General

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described active ingredients prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of inflammatory disorders.

The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression.

Conversely, the term “treatment” refers to the clinical application of active agents to combat an already existing condition whose clinical presentation has already been realized in a patient.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb. Other terms as used herein are meant to be defined by their well-known meanings in the art. Unless specifically stated or obvious from context, as used herein, the term “or” is understood to be inclusive.

Throughout this specification and claims, the word “comprise” or variations such as “comprises” or “comprising” indicate the inclusion of any recited integer or group of integers but not the exclusion of any other integer or group of integers.

As used herein, the term “consists essentially of” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises” “comprising”, and “having” are/is interchangeable with “consisting”.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### EXAMPLES

Generally, the nomenclature used herein, and the laboratory procedures utilized in the present invention include molecular, biochemical, and microbiological techniques. Such techniques are thoroughly explained in the literature. As used hereinthroughout, the term Cpd. 1, Cpd. 2, Cpd. 3 etc., refers to a compound indicated by a number (e.g. 1, 2, 3 etc.) within the synthetic scheme.

### Materials and Methods

Cell line: Shh Light 2 cell line which is based on NIH3T3 mouse fibroblast cell line genetically modified to stably express the Firefly Luciferase reporter gene under the transcription control of Gli 1 binding sequence and the Renila Luciferase gene under a control pRL-SV40 promoter was licensed from Johns Hopkins University (JHU).

Cell culture: Cells are cultured in Complete Medium [formulated as DMEM, 10% FBS, 1% sodium pyruvate, 1% L-glutamine, 0.4 mg/ml G418, 0.15 mg/ml Zeocin] until 80% confluence is achieved. Once sufficient confluency is achieved, cells are detached from the 10 cm culture dish using trypsin-EDTA, washed in Growth Medium [formulated as DMEM, 10% FBS, 1% sodium pyruvate, 1% L-glutamine, 1% PenStrep] and resuspended in fresh Growth Medium at a concentration of 4.3*10⁵ cells/ml. To a white 96-well plate 100 ml of cells suspension is transferred and incubated in a humified incubator at 37° C. with 5% CO₂ overnight until the cells reach confluency the following day.

On the following day, a 13 point 1:1 dilution of a stock 20 mM Gli 1 inhibitor compound in DMSO is performed and 1.5 ml of each diluted Gli1 inhibitor compound is diluted in 240 ml Assay Medium [formulated as DMEM, 2% FBS, 1% sodium pyruvate, 1% L-glutamine, 1% PenStrep]. The Growth Medium is removed from the cell-plated 96-well plate and 60 ml of the diluted Gli1 inhibitor is transferred to each well and plates are incubated at 37° C. and 5% CO₂ for 30 minutes.

Then, the plates are removed from incubator and Purmorphamine (a Smoothened agonist to induce Hedgehog signaling and Gli1 activation in cells) at a final concentration of 1.5 mM is added to each well and the plates are reinserted to the incubator for 24 hours incubation. The following day, Firefly and Renila Luciferase levels are measured using the Dual-Glo Luciferase system and the ratio between the Firefly and Renila is calculated for normalization of signal. The ratio of the signal in the treated cells is plotted vs. compound concentration and the IC₅₀ value of each compound is calculated by generating a non-linear regression curve using the 4PL function in GraphPad Prism 8 software.

### Example 1

### Gli1-Luciferase Reporter Assay

In order to select the most potent Gli1 inhibitor analog, an assay that can quantify the potency of small molecule Gli1 inhibitor should be developed. The primary screening assay is based on a NIH3T3 mouse fibroblast cell line genetically modified to stably express the Firefly Luciferase reporter gene under the transcription control of Gli1 binding sequence and the Renila Luciferase gene under a control pRL-SV40 promoter. This assay allows the determination of a compound's IC₅₀ value in inhibiting Gli1 activity.

The compounds of the invention are currently undergoing biological studies, in order to estimate their potency (IC₅₀ value) with respect to Gli1 inhibiting activity. Several compounds showed significant Gli1 inhibition, compared to a negative control, showing sub-micromolar IC₅₀ values. Some compound exhibited IC₅₀ values between 0.1 and 10 nM. Microsomal assay was performed as described hereinbelow.

### Test Compound and Control Working Solution Preparation:

Working solution: 5 μL of compound and control stock solution (10 mM in dimethyl sulfoxide (DMSO)) were diluted with 495 μL of acetonitrile (ACN) (intermediate solution concentration: 100 μM, 99% ACN)

### NADPH Cofactor Preparation:

NADPH powder: β-Nicotinamide adenine dinucleotide phosphate reduced form, tetrasodium salt; NADPH·4Na (Vendor: BONTAC). The appropriate amount of NADPH powder was weighed and diluted into a 10 mM MgCl2 solution (working solution concentration: 10 unit/mL; final concentration in reaction system: 1 unit/mL). The results are summarized in Table 1.

TABLE 1

| | Luciferase cell |
|---|---|
| Compound | based IC50 (nM) |
| Glx-088RS | 7.1 |
| Glx-088P1 | 0.78 |
| Glx-088P3 | 0.18 |
| Glx-308 | 55 |
| Glx-322 | 12 |
| Glx-341 | 16.7 |
| Glx-344 | 385 |
| Glx-344A | 172 |
| Glx-369 | 73 |
| Glx-402 | 430 |
| Glx-530P2 | 287 |

Microsomal half-life of the presented compounds was characterized in mouse and humans. Most compounds were stable presenting a microsomal (human and/or mice) half-life between 0.5 min and 40 min.

In addition, some benzyl series compounds such as Compound 9, 10, 12, 13, 16 and 17 exhibited in-vitro efficacy, with IC 50 of between 0.1 and 100 nM. Compound 10 and Compound 12 are highly potent (IC₅₀=16.6 and 5.6 nM, respectively) benzyl series compounds.

### Example 2

### Efficacy EAE Model

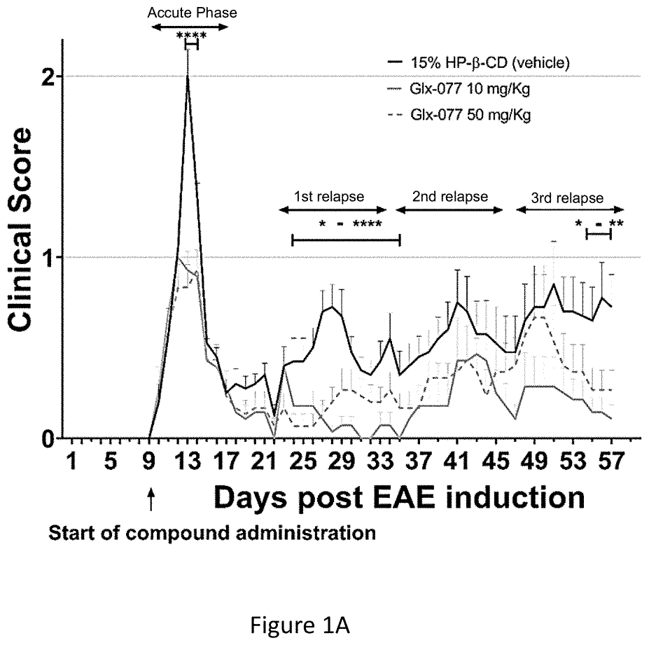

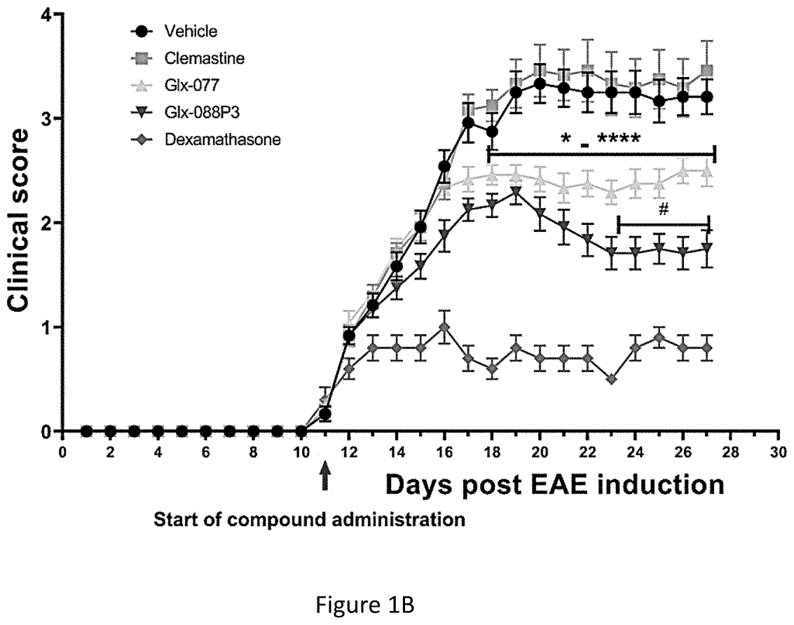

Eight-weeks old female SJL/L mice were immunized on Day 1 with an emulsion of PLP₁₃₉₋₁₅₁/CFA and injected with pertussis toxin to induce disease. At the start of clinical signs (Days 9-10), the animals were administered with compound 6 (Glx-077) at a daily dose of 10 mg/Kg or 50 mg/Kg, or with a vehicle (15% HP-β-CD in water) until completion of trial (Day 57). A graph, summarizing the results of this experiment is represented in FIG. 1A.

Thirteen-weeks old female C57B16 mice were immunized on Day 1 with an emulsion of MOG₃₅₋₅₅/CFA and injected with pertussis toxin to induce disease. At the start of clinical signs (Days 9-10), the animals were daily administered with Glx-077, Glx-088P3, clemastine fumarate (at 10 mg/Kg) or vehicle (15% HP-β-CD in water) until completion of experiment (Day 28). Mice administered with dexamethasone were used as a positive control group. Clinical score of the mice was measured daily. A graph, summarizing the results of this experiment is represented in FIG. 1B.

### Example 3

### Pharmacokinetic Study

Compounds were formulated in 15% HP-b-CD in water (IV formulation was at pH=5.0 and PO was at pH=3.0). Blood was obtained from facial vein pre-dosing and at 0.083, 0.25, 0.5, 1, 2, 4, 8, 24 hours post dosing for a total of 9 time-points, semi-serial bleeding. For PO dosing, another group of animals (n=3) were terminated at 2 hours post dosing and brains were extracted to analyze brain exposure and calculate brain/plasma ratio. Lower limit of quantification (LLOQ) for brain and plasma samples was 1 ng/ml. Table 2 summarizes the results of the experiment.

TABLE 2

| | | | | | | Brain levels | Brain/ | |
|---|---|---|---|---|---|---|---|---|
| | | | | | | 2 h post PO | plasma | |
| | Route of | Dose | T½ | | AUC_{INF-plasma} | administration | ratio | |
| Compound | administration | (mg/Kg) | (hr) | Cmax | (ng/ml*hr) | (ng/g) | (2 h)% | % F |
| Glx-088RS | PO | 10 | 1.99 | 698 | 748 | 211 | 129 | ND |
| Glx-088P3 | IV | 2 | 1.53 | NA | 517 | ND | ND | NA |
| | PO | 10 | 1.5 | 268 | 517 | 123 | 208 | 38 |
| | | 30 | 1.69 | 1199 | 2674 | 1104 | 236 | 65.6 |

### Example 4

### Synthetic Procedures

An exemplary synthetic procedure for preparation of Glx-265 is as follows:

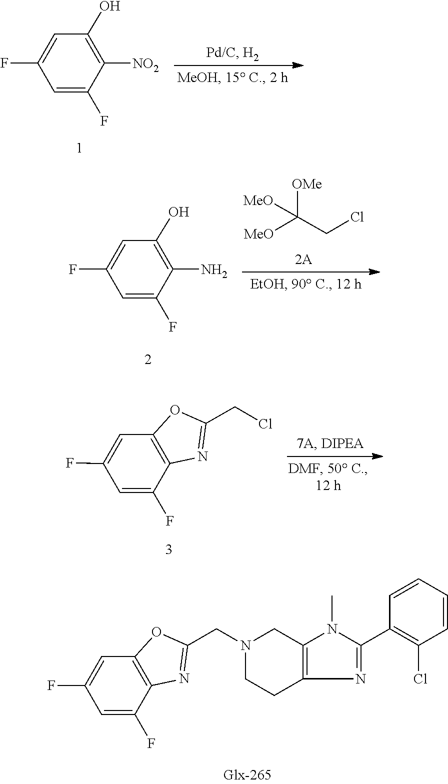 [SMILES: O=[N+]([O-])c1c(O)cc(F)cc1F ; Nc1c(O)cc(F)cc1F ; COC(CCl)(OC)OC ; Fc1cc(F)c2nc(CCl)oc2c1 ; Cn1c(-c2ccccc2Cl)nc2c1CN(Cc1nc3c(F)cc(F)cc3o1)CC2]

General procedure for preparation of Glx-265: To a mixture of 2-(chloromethyl)-4,6-difluoro-1,3-benzoxazole (100 mg, 491.22 umol, 1 eq) and 2-(2-chlorophenyl)-3-methyl-4,5,6,7-tetrahydroimidazo[4,5-c]pyridine (97.72 mg, 343.86 umol, 0.7 eq, HCl) in DMF (3 mL) was added DIPEA (190.46 mg, 1.47 mmol, 256.69 uL, 3 eq) in one portion at 15° C. under N₂. The mixture was heated to 50° C. and stirred for 12 hours. The reaction mixture was filtered. The filtrate was purified by prep-HPLC.

An exemplary synthetic procedure for preparation of Glx-077 is as follows:

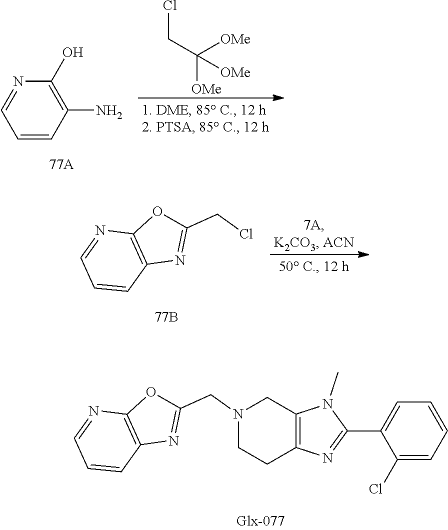 [SMILES: Nc1cccnc1O ; COC(CCl)(OC)OC ; ClCc1nc2cccnc2o1 ; "7A,K2CO3, ACN" ; Cn1c(-c2ccccc2Cl)nc2c1CN(Cc1nc3cccnc3o1)CC2]

An exemplary synthetic procedure for preparation of Glx-88 (R and S) is as follows:

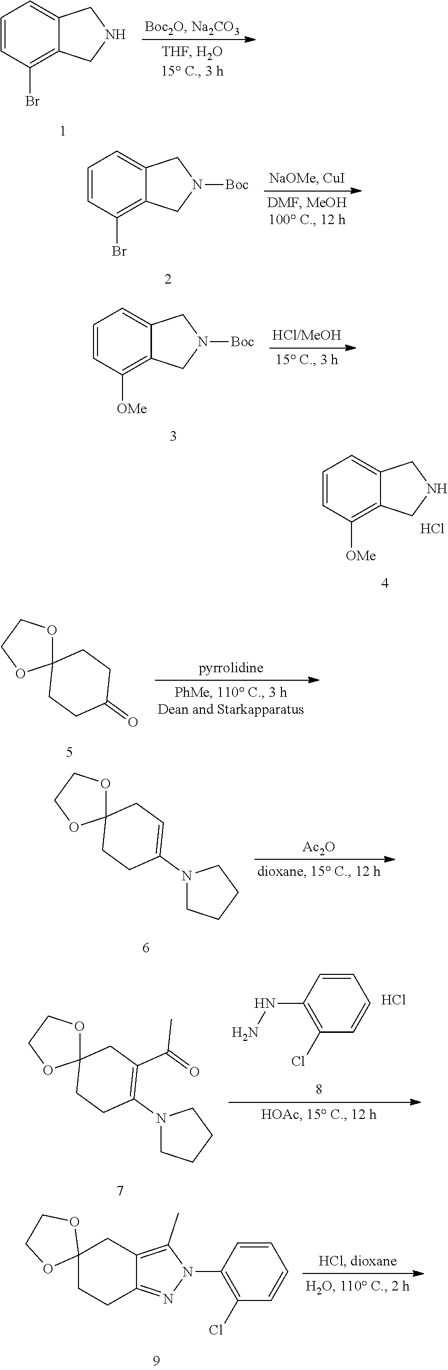

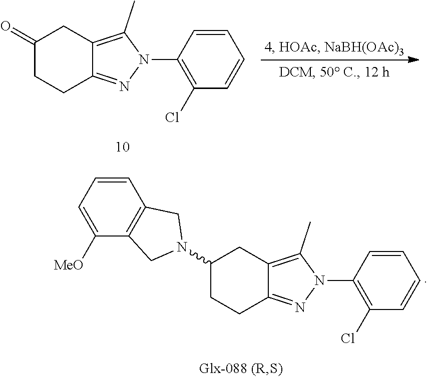 [SMILES: Cc1c2c(nn1-c1ccccc1Cl)CCC(=O)C2 ; COc1cccc2c1CN(C1CCc3nn(-c4ccccc4Cl)c(C)c3C1)C2]

General procedure for preparation of Glx-088(R)&Glx-088(S): To a solution of 2-(2-chlorophenyl)-3-methyl-6,7-dihydro-4H-indazol-5-one (0.4 g, 1.53 mmol, 1 eq) and 4-methoxyisoindoline (228.89 mg, 1.23 mmol, 8.04e-1 eq, HCl) in DCM (20 mL) was added HOAc (92.13 mg, 1.53 mmol, 87.75 uL, 1 eq) and NaBH(OAc)₃ (975.49 mg, 4.60 mmol, 3 eq). The mixture was stirred for 12 h at 50° C. The reaction mixture was quenched by addition H₂O 50 mL and extracted with EtOAc (50 mL*4). The combined organic layers were concentrated under reduced pressure to give a residue. The residue was purified by prep-HPLC. An exemplary synthetic procedure for preparation of Glx-308 is as follows:

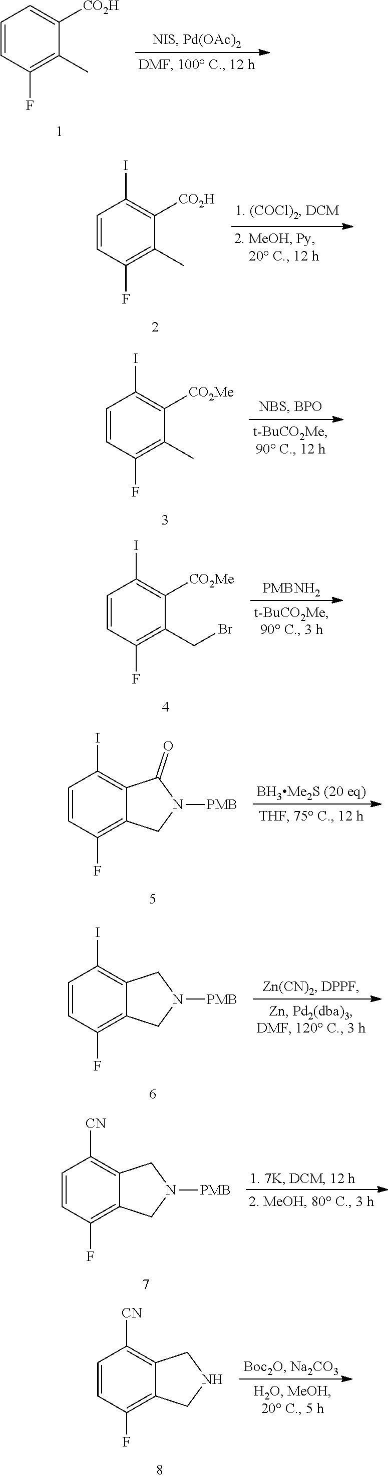

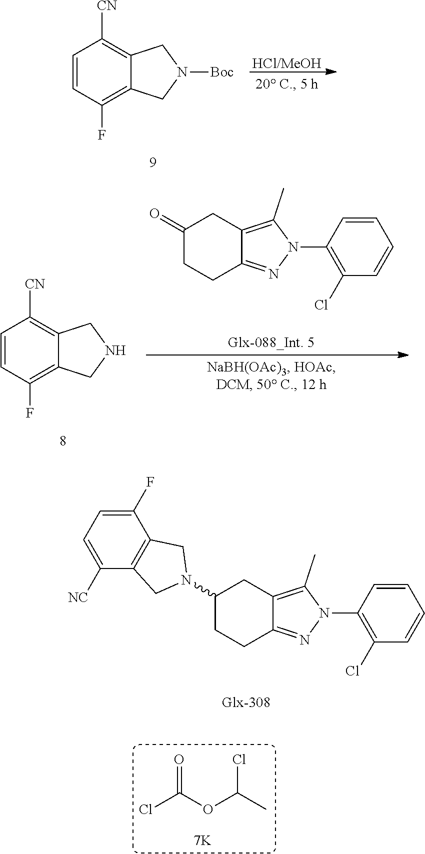

An exemplary synthetic procedure for preparation of Glx-322 is as follows:

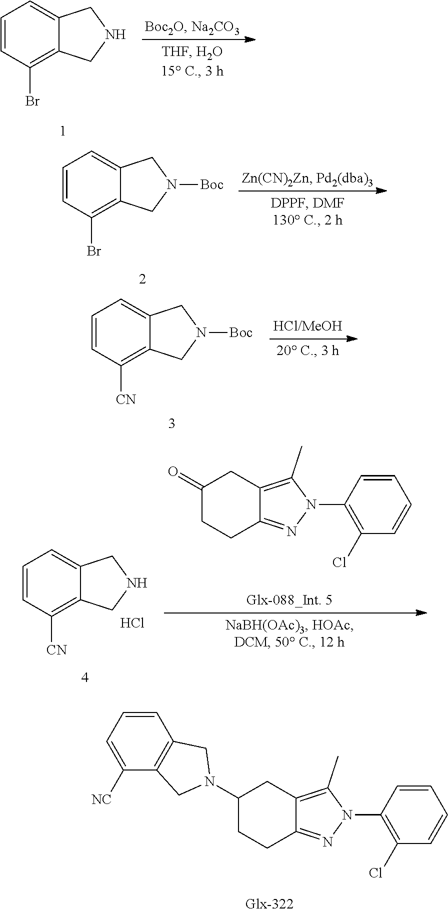

An exemplary synthetic procedure for preparation of Glx-341 is as follows:

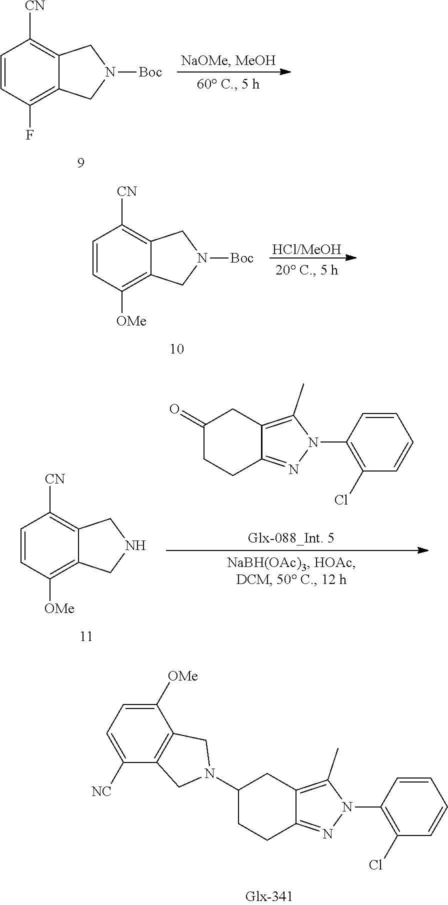

An exemplary synthetic procedure for preparation of Glx-344 and 344A is as follows:

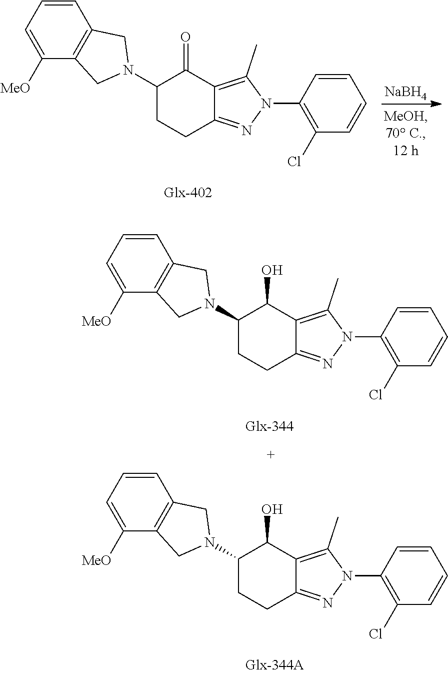

An exemplary synthetic procedure for preparation of Glx-369 is as follows:

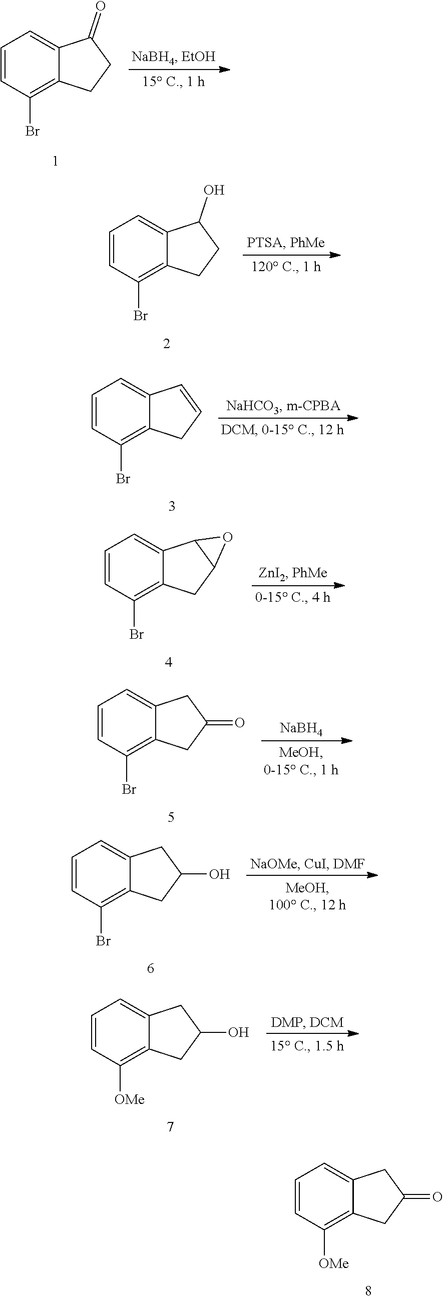

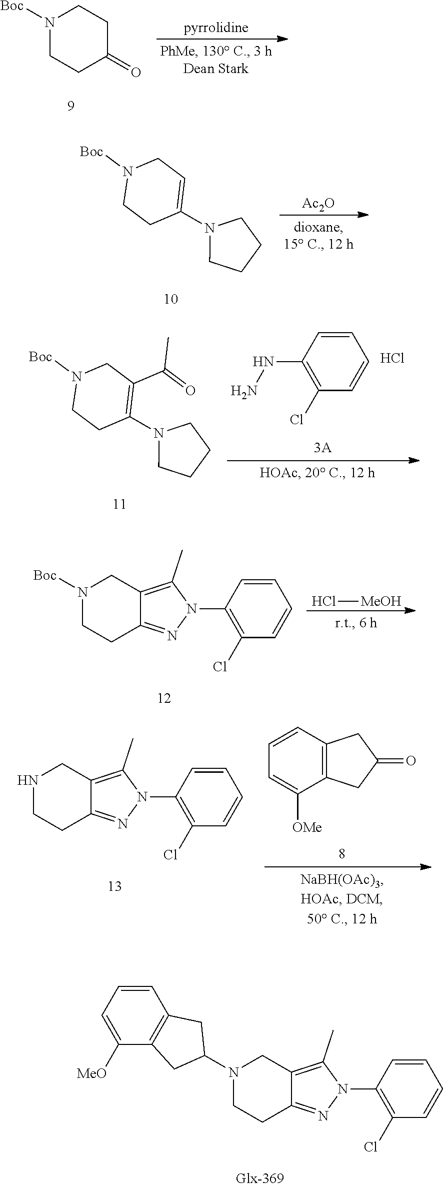

An exemplary synthetic procedure for preparation of Glx-402 is as follows:

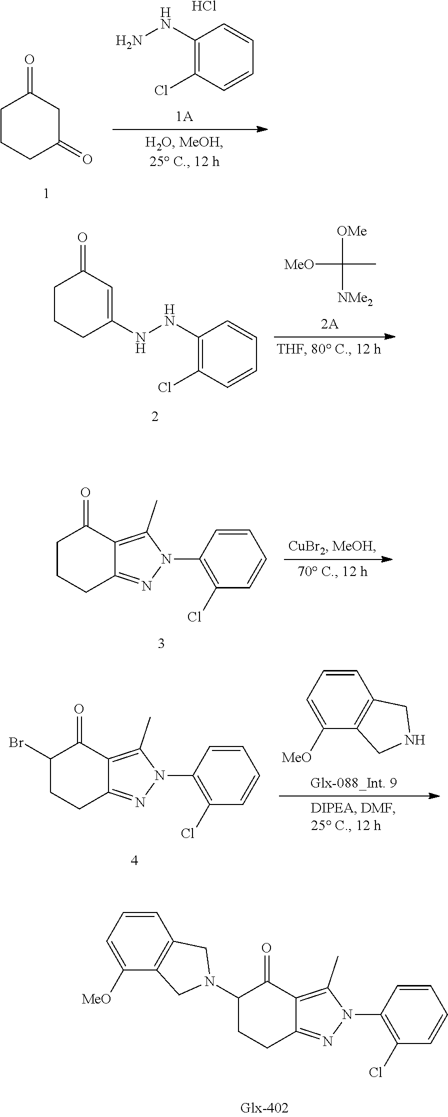

An exemplary synthetic procedure for preparation of Glx-367 is as follows:

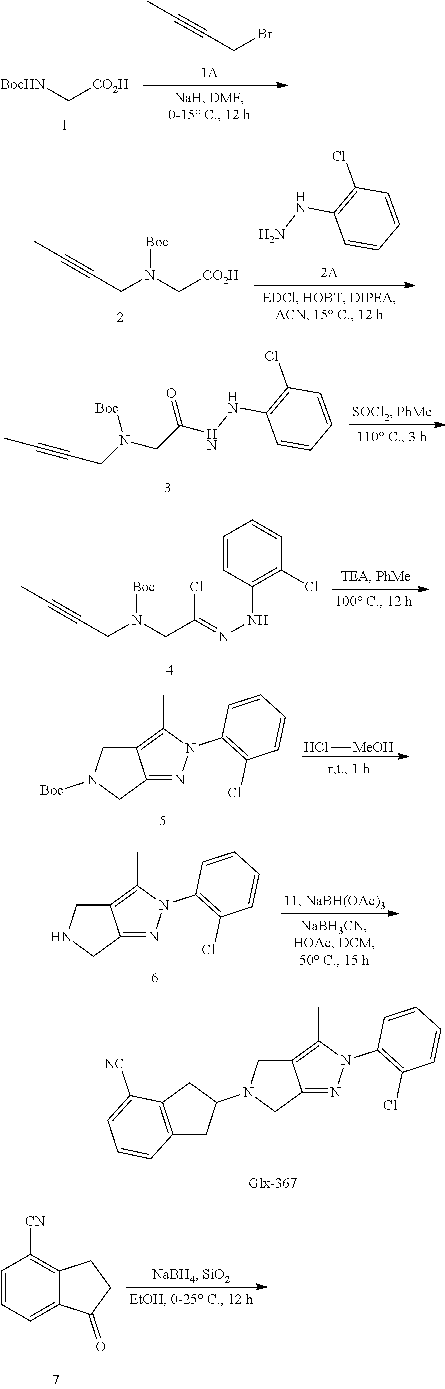

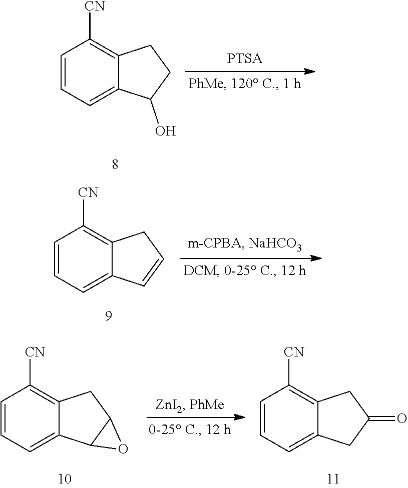 [SMILES: N#Cc1cccc2c1CCC2O ; N#Cc1cccc2c1CC=C2 ; N#Cc1cccc2c1CC1OC21 ; N#Cc1cccc2c1CC(=O)C2]

An exemplary synthetic procedure for preparation of Glx-530, Glx-530P1 and Glx-530P2 is as follows:

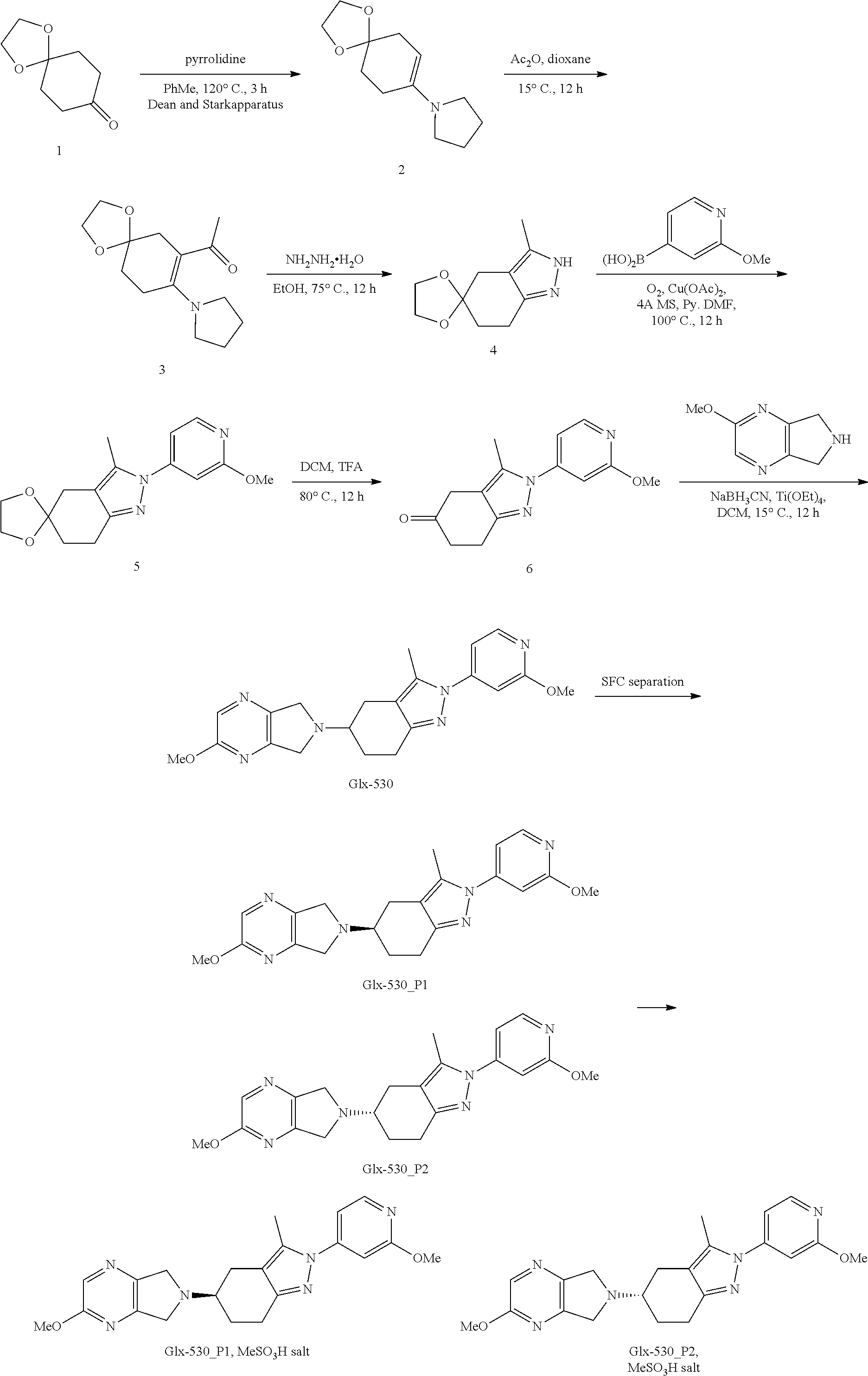

General procedure for preparation of Glx-530: To a solution of 2-(2-methoxy pyridyl)-3-methyl-6,7-dihydro-4H-indazol-5-one (1 eq) and 3-methoxy-6,7-dihydro-5H-pyrrolo[3,4-b]pyrazine (1 eq, HCl) in DCM (20 mL) was added Ti(OEt)₄ (1 eq) at 15° C. After addition, the mixture was stirred at this temperature for 1 hr, and then NaBH₃CN (3 eq) was added at 15° C. The resulting mixture was stirred at 15° C. for 11 hr. The residue was purified by prep-HPLC.

SFC separation method: The residue was purified by SFC (column: DAICEL CHIRALPAK AD (250 mm*30 mm, 10 um); mobile phase: [Neu-ETOH]; B %: 55%-55%, 8 min).

Exemplary procedures for preparation of the preparation of HCL/MeS_{O3}H salts: To a solution of the desired compound methane sulfonic acid or HCL is added and the mixture stirred at 15° C.-20° C. The reaction mixture is lyophilized affording the desired salt. Additional compounds disclosed herein, can be synthesized based on the synthetic procedures described hereinabove, by using synthetic methods well-known in the art. Further synthetic procedures are incorporated herein.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A compound or a salt thereof, wherein said compound is represented by Formula VIIIa: 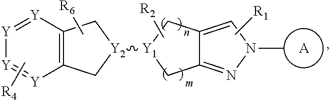
wherein:
A comprises optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof;
each R₁, R₂ and R₆ represents one or more substituents independently comprising hydrogen, halogen, —NO₂, —CN, optionally substituted C₁-C₆ alkyl, C₁-C₆ haloalkyl, —NH₂, —NH(C₁-C₆ alkyl), —N(C₁-C₆ alkyl)₂, carbonyl, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, and optionally substituted C₃-C₈ cycloalkyl or a combination thereof, or each R₁, and R₂ independently represents hydrogen;
R₄ represents one or more substituents each independently comprising hydrogen, halogen, —NO₂, —CN, C₁-C₆ alkyl optionally substituted with one or more R₅, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, optionally substituted C₃-C₈ cycloalkyl, optionally substituted heterocyclyl, optionally substituted aryloxy, optionally substituted heteroaryloxy, optionally substituted cycloalkyloxy, 2-hydroxy-3-methoxypropoxy, (2-methoxyethoxy)methyl, and 2-(3-(but-3-yn-1-yl)-3H-diazirin-3-yl)ethoxy or a combination thereof;
each R₅ independently comprises hydrogen, deuterium, C₁-C₆ alkyl, C₁-C₆ haloalkyl, optionally substituted C₃-C₈ cycloalkyl, —CN, —CONRR, or a combination thereof;
each R independently comprises hydrogen, C₁-C₆ alkyl, tolyl, optionally substituted phenyl, optionally substituted benzyl or a combination thereof;
Y, Y₁ and Y₂ independently comprises CH, C, N, or NH; and
each m and n is independently 1 or 2.

2. The compound of claim 1, wherein said compound is represented by or comprises Formula VIIIa: 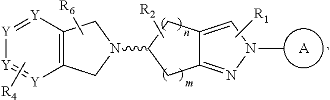
or formula VIIIb:

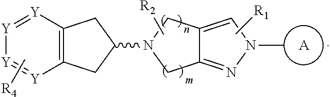

3. The compound of claim 1, wherein A comprises C₅-C₆ aryl optionally substituted with one or more R₃, C₅-C₆ heteroaryl optionally substituted with one or more R₃, or C₄-C₈ cycloalkyl optionally substituted with one or more R₃ or a combination thereof; wherein R₃ is selected from the group comprising hydrogen, halogen, —NO₂, —CN, optionally substituted C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂, cyclopropylethynyl, optionally substituted aryl, optionally substituted heteroaryl, optionally substituted heterocyclyl, and optionally substituted C₃-C₈ cycloalkyl or a combination thereof.

4. The compound of claim 1, wherein A comprises any of: 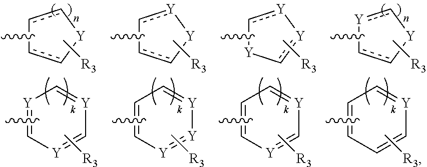
wherein k is 0 or 1 and n is 1 or 2.

5. The compound of claim 1, wherein R₂ represents a substituent selected from the group comprising hydrogen, halogen, —NO₂, —CN, optionally substituted C₁-C₆ alkyl, C₁-C₆ haloalkyl, —NH₂, —NH(C₁-C₆ alkyl), —N(C₁-C₆ alkyl)₂, carbonyl, —OH, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH₂, —CONH(C₁-C₆ alkyl), —CON(C₁-C₆ alkyl)₂, —CONH—OH, —CO₂H, —CO₂(C₁-C₆ alkyl), —SO₂R, —SO₂OR, —SO₂N(R)₂.

6. The compound of claim 1, wherein A comprises C₅₋₆ aryl optionally substituted with one or more R₃ or C₅₋₆ heteroaryl optionally substituted with one or more R₃, optionally wherein R₁ represents a substituent independently comprising hydrogen, optionally substituted C₁-C₄ alkyl.

7. (canceled)

8. The compound of claim 1, wherein said compound is represented by or comprises any one of Formula IX:

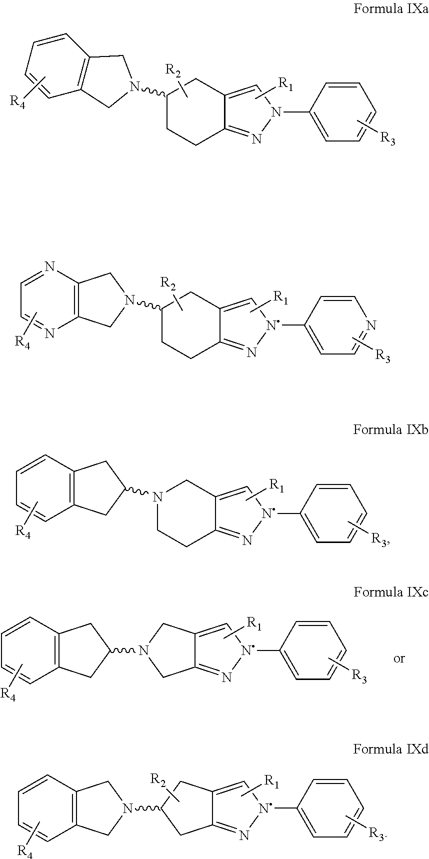

9. The compound of claim 1, wherein said compound is represented by or comprises any one of Formula X:

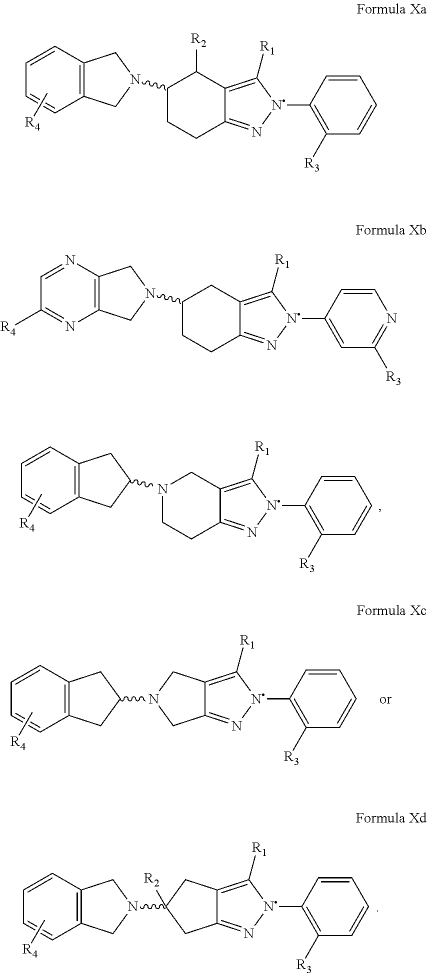

10. The compound of claim 1, wherein R₁ represents a substituent selected from the group comprising methyl, —NH₂ or —CN, wherein each R₂, R₃ and R₄ independently represents a substituent selected from the group comprising halogen, carbonyl, —CN, —OMe, —OH, any combination thereof, or is absent; optionally wherein said compound is stable in an aqueous solution for at least 1 hour.

11. (canceled)

12. (canceled)

13. A pharmaceutical composition, comprising the compound of claim 1 and a pharmaceutically acceptable carrier.

14. (canceled)

15. (canceled)

16. (canceled)

17. (canceled)

18. A method for preventing or treating a disease or a disorder associated with an abnormal expression of Gli in a subject, comprising administering to the subject a pharmaceutical composition comprising the compound of claim 1, thereby preventing or treating a disease or a disorder associated with an abnormal expression of Gli in a subject.

19. The method of claim 18, wherein said disease or a disorder is a neurological disorder or cancer; optionally wherein said cancer is characterized by Gli expression or Gli over-expression.

20. The method of claim 18, comprising the step of selecting the subject as: (a) afflicted with a disease or a disorder comprising an abnormal expression of Gli; or (b) afflicted said cancerous cell expressing Gli or abnormal expressing of Gli.

21. (canceled)

22. The method of claim 18, wherein said cancer is selected from the group comprising: breast cancer, pancreatic cancer, colon cancer, lung cancer, rhabdomyosarcoma, basal-cell carcinoma, glioblastoma, medulloblastoma, leukemia, prostate cancer, skin cancer, lymphoma, esophageal cancer, ovarian cancer, thyroid cancer, osteosarcoma, liver cancer, multiple endocrine neoplasia, gastrointestinal cancer, or mesothelioma; and wherein said neurological disorder is selected from the group comprising: multiple sclerosis, central pontine myelinolysis, acute disseminated encephalomyelitis, progressive multifocal leukoencephalopathy, subacute sclerosing panencephalitis, post-infectious encephalomyelitis, chronic inflammatory demyelinating polyneuropathy, Devic's disease, Balo's concentric sclerosis, the leukodystrophies, optic neuritis, transverse myelitis, cerebral palsy, spinal cord injury, age-associated myelin deficiency, Alzheimer's Disease, and acquired and inherited neuropathy in the peripheral nervous system.

23. (canceled)

24. The method of claim 18, wherein said preventing or said treating comprises inhibiting Gli function in said cell.

25. The method of claim 18, wherein said administering is by an oral administration, a topical administration, a systemic administration or any combination thereof.

26. The method of claim 18, wherein said Gli is Gli1.
